(19) United States
(12) Patent Application Publication
(10) Pub. No.: US 2026/0201337 A1
(43) Pub. Date: Jul. 16, 2026

(54) PRE-LICENSED COMPOSITION AND CELL CULTURE METHODS

(71) Applicant: Mesoblast International Sarl, Meyrin (CH)

(72) Inventors: Silviu ITESCU, Melbourne (AU); Paul SIMMONS, Melbourne (AU); Jack HAYES, New York, NY (US); Justin HORST, New York, NY (US); Fiona SEE, Melbourne, Victoria (AU)

(21) Appl. No.: 19/137,213

(22) PCT Filed: Dec. 8, 2023
(86) PCT No.: PCT/IB2023/062429

§ 371 Date: Jun. 9, 2025

Related U.S. Application Data
(60) Provisional application No. 63/386,876, filed on Dec. 9, 2022.
(60) Provisional application No. 63/507,009, filed on Jun. 8, 2023.

(51) Int. Cl. C12N 5/0775

(52) U.S. Cl. CPC C12N 5/0662; C12N 2501/2306; C12N 2501/2308; C12N 2501/2317; C12N 2501/24

(57) ABSTRACT

The present disclosure relates to MLPSC populations and methods and cell culture media for producing the same. Such methods and media may be particularly useful for promoting pre-licensing of mesenchymal lineage precursor or stem cells (MLPSCs.

### TECHNICAL FIELD

The present disclosure relates to MLPSC populations and methods and cell culture media for producing the same. Such methods and media may be particularly useful for promoting pre-licensing of mesenchymal lineage precursor or stem cells (MLPSCs).

### BACKGROUND

MLPSCs, e.g., multipotent mesenchymal stem cells (MSCs) have been proposed as an attractive candidate for therapeutic applications because of their high proliferation and differentiation potential as well as immunoregulatory/anti-inflammatory and other beneficial properties (Caplan A I (2007) J. Cell Physiol., 213, 341-347; Prockop D J (2007) Clin Pharmacol Ther., 82, 241-243). Ex vivo propagation of sparse populations of mesenchymal stem cells (MSC) is often necessary for generating numbers suitable for therapeutic applications.

Conventional media used for isolating and expanding MSC consist of a defined basal medium (e.g., Dulbecco's modified Eagle's medium (DMEM) or α-modified minimum essential medium (α-MEM)) supplemented with fetal bovine serum (FBS) because of its high content of stimulatory growth factors. Although these media are generally reported to support the proliferation of MLPSCs for multiple passages, batch-to-batch variation in FBS could cause inconsistencies in the therapeutic properties or potency of MLPSCs.

Accordingly, there remains an unmet need for compositions and methods for consistent production of therapeutically effective MLPSCs in serum-containing cell culture.

### SUMMARY

The present inventors have found that MLPSC cultures supplemented with newborn serum unexpectedly exhibit a pre-adapted (AKA “pre-licensed”) anti-inflammatory state that increases their therapeutic efficacy, particularly in the context of persistent inflammation. For example, newborn calf serum (NBCS) is generally marketed as an equivalent/acceptable substitute for fetal bovine serum (FBS). However, the present inventors unexpectedly found that this was not the case, as NBCS supplementation induced a pre-licensing effect on cultured MLPSCs as described herein. Analysis of the newborn serum used to culture MLPSCs with increased therapeutic efficacy surprisingly revealed increased levels of cytokines, in particular, in regard to cytokines where a corresponding receptor is expressed by MLPSCs. These findings underpin the generation of novel MLPSC compositions through culture expansion with certain pro-inflammatory cytokines and/or in newborn serum.

Accordingly, in one aspect, the present disclosure relates to a composition comprising a culture-expanded population of mesenchymal lineage precursor or stem cells (MLPSCs), wherein the MLPSCs have been culture expanded in media containing Interferon (IFN)-gamma and/or tumor necrosis factor (TNF)-alpha, wherein the level(s) of IFN-gamma and/or TNF-alpha in the media are <1 ng/ml. For example, the level of IFN-gamma may be <500 pg/ml. In an example, the level of IFN-gamma is <100 pg/ml. In an example, the level of TNF-alpha is <750 pg/ml. In another example, the level of TNF-alpha is <500 pg/ml. In an example, the levels of IFN-gamma and TNF-alpha are both <500 pg/ml.

In another aspect, the present disclosure relates to a composition comprising a culture-expanded population of mesenchymal lineage precursor or stem cells (MLPSCs), wherein the MLPSCs were culture expanded in media containing one or more pro-inflammatory cytokines selected from the group consisting of IL-6; IL-8; IL-17A; MCP-1; MIP-1-alpha; MIP-1-beta; IP-10,

Accordingly, in another aspect, the present disclosure relates to a composition comprising:
(i) a culture-expanded population of mesenchymal lineage precursor or stem cells (MLPSCs), wherein the MLPSCs have been culture expanded in media containing:
   IFN-gamma and/or TNF-alpha; and/or,
   one or more pro-inflammatory cytokines selected from the group consisting of IL-6; IL-8; IL-17A; MCP-1; MIP-1-alpha; MIP-1-beta; IP-10.

In an example, the media contains three or more pro-inflammatory cytokines.

In another example, the media contains two or more pro-inflammatory cytokines selected from the group consisting of IL-6; IL-8; IL-17A; MCP-1; MIP-1 -alpha; MIP-1-beta; IP-10.

In another example, the media contains IL-6. In another example, the media contains IL-8 and/or IL-17A. In another example, the media contains IFN-gamma and TNF-alpha. For example, the media may contain IFN-gamma, TNF-alpha and, one or more pro-inflammatory cytokines selected from the group consisting of IL-6; IL-8; IL-17A; MCP-1; MIP-1-alpha; MIP-1-beta; IP-10.

In an example, the level of IFN-gamma is <1 ng/ml. For example, the level of IFN-gamma may be <500 pg/ml. In an example, the level of IFN-gamma is <100 pg/ml. In another example, the level of TNF-alpha is <1 ng/ml. For example, the level of TNF-alpha may be <750 pg/ml. In an example, the level of TNF-alpha is <400 pg/ml.

In another example, the media contains serum which comprises the pro-inflammatory cytokines. In an example, the serum is newborn mammalian serum. For example, the serum may be newborn calf serum. In an example, the newborn serum is obtained no more than 21 days after birth.

In an example, a composition of the disclosure is cryopreserved.

In an example, the media is characterised by one or more or all of the following:
a level of IFN-gamma greater than 1 pg/ml;
a level of TNF-alpha greater than 2 pg/ml;
a level of IL-6 greater than 3 pg/ml;
a level of IL-8 greater than 500 pg/ml;
a level of IL-17A greater than 0.2 pg/ml;
a level of MCP-1 greater than 3 pg/ml;
a level of MIP-1-alpha greater than 0.5 pg/ml;
a level of MIP-1-beta greater than 3 pg/ml;
a level of IP-10 greater than 500 pg/ml.

In an example, the media is characterised by one or more or all of the following:
a level of IFN-gamma between 1 pg/ml and <1 ng/ml;
a level of TNF-alpha between than 2 pg/ml and <1 ng/ml;
a level of IL-6 greater than 3 pg/ml;
a level of IL-8 greater than 500 pg/ml;
a level of IL-17A greater than 0.2 pg/ml;
a level of MCP-1 greater than 3 pg/ml;
a level of MIP-1-alpha greater than 0.5 pg/ml;
a level of MIP-1-beta greater than 3 pg/ml;
a level of IP-10 greater than 500 pg/ml.

In another example, the media is characterised by a level of IFN-gamma between 1 pg/ml and <1 ng/ml; a level of TNF-alpha between than 2 pg/ml and <1 ng/ml; and, one or more or all of the following:
a level of IFN-gamma between 1 pg/ml and <1 ng/ml;
a level of TNF-alpha between than 2 pg/ml and <1 ng/ml;
a level of IL-6 greater than 3 pg/ml;
a level of IL-8 greater than 500 pg/ml;
a level of IL-17A greater than 0.2 pg/ml;
a level of MCP-1 greater than 3 pg/ml;
a level of MIP-1-alpha greater than 0.5 pg/ml;
a level of MIP-1-beta greater than 3 pg/ml;
a level of IP-10 greater than 500 pg/ml.

In another example, the media is characterised by supplementation with serum comprising one or more or all of the following:
a level of IFN-gamma greater than 10 pg/ml;
a level of TNF-alpha greater than 20 pg/ml;
a level of IL-6 greater than 30 pg/ml;
a level of IL-8 greater than 5,000 pg/ml;
a level of IL-17A greater than 2 pg/ml;
a level of MCP-1 greater than 30 pg/ml;
a level of MIP-1-alpha greater than 5 pg/ml;
a level of MIP-1-beta greater than 30 pg/ml;
a level of IP-10 greater than 5,000 pg/ml.

In an example, the media comprises IL-10. In another example, the media comprises IL-36RA. In another example, the media comprises IL-10 and IL-36RA. In an example, the level of IL-10 is greater than 0.3 pg/ml. For example, the level of IL-10 may be greater than 30 pg/ml. In an example, the level of IL-10 is greater than 400 pg/ml. In an example, the level of IL-36RA is greater than 50 pg/ml.

In an example, the media comprises at least 5% (v/v) newborn mammalian serum. In another example, the media comprises 5% (v/v) newborn mammalian serum. In another example, the media is serum free.

Accordingly, in another aspect, the present disclosure relates to a composition for pre-licensing MLPSCs, comprising: (i) a human cell population enriched for MLPSCs; and (ii) serum-containing cell culture medium; wherein the serum is a serum comprising one or more pro-inflammatory cytokines.

In another aspect, the present disclosure relates to a composition for pre-licensing and cryopreservation of MLPSCs, comprising: (i) a human cell population enriched for MLPSCs; (ii) serum comprising one or more pro-inflammatory cytokines; and (iii) a cryopreservative. In an example, the MLPSCs are cryopreserved at least twice.

In another aspect, the present disclosure relates to a cell culture medium suitable for proliferation and pre-licensing of MLPSCs, the cell culture medium comprising serum containing one or more pro-inflammatory cytokines.

In another aspect, the present disclosure relates to an in vitro method for pre-licensing human mesenchymal lineage precursor or stem cells (MLPSCs), the method comprising culturing the MLPSCs in media containing:
IFN-gamma and/or TNF-alpha; and/or, one or more pro-inflammatory cytokines selected from the group consisting of IL-6; IL-8; IL-17A; MCP-1; MIP-1-alpha; MIP-1-beta; IP-10; and/or,
newborn mammalian serum obtained no more than 21 days after birth.

For example, the method may comprise culturing the MLPSCs in media containing IFN-gamma and TNF-alpha. In an example, the level(s) of IFN-gamma and/or TNF-alpha are <1 ng. In another example, the method comprises culturing the MLPSCs in culture media which comprises one or more pro-inflammatory cytokines selected from the group consisting of IL-6; IL-8; IL-17A; MCP-1; MIP-1-alpha; MIP-1-beta; IP-10. In another example, the method comprises culturing the MLPSCs in culture medium which comprises newborn mammalian serum obtained no more than 21 days after birth.

In an example, the method comprises culturing the MLPSCs in media containing IFN-gamma and/or TNF-alpha; and, one or more pro-inflammatory cytokines selected from the group consisting of IL-6; IL-8; IL-17A; MCP-1; MIP-1-alpha; MIP-1-beta; IP-10.

In an example, the media contains three or more pro-inflammatory cytokines. In this example, two or more of the pro-inflammatory cytokines may be selected from the group consisting of IL-6; IL-8; IL-17A; MCP-1; MIP-1-alpha; MIP-1-beta; IP-10. In an example, the media contains IL-6. In an example, the media contains IL-8 and/or IL-17A. In an example, the media contains IFN-gamma and TNF-alpha. In an example, the level of IFN-gamma is <500 pg/ml. In an example, the level of IFN-gamma is <100 pg/ml. In another example, the level of TNF-alpha is <750 pg/ml. In an example, the level of TNF-alpha is <400 pg/ml.

In an example, the media contains serum which comprises the pro-inflammatory cytokines. For example, the serum may be a newborn serum such as newborn calf serum. In an example, the newborn serum is obtained no more than 21 days after birth.

In an example, the media is characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml

In an example, the media comprises IL-10. In another example, the media comprises IL-36RA. In another example, the media comprises IL-10 and IL-36RA. In an example, the level of IL-10 is greater than 0.3 pg/ml. For example, the level of IL-10 may be greater than 30 pg/ml. In an example, the level of IL-10 is greater than 400 pg/ml. In an example, the level of IL-36RA is greater than 50 pg/ml.

In an example, the media comprises at least 5% (v/v) newborn mammalian serum. In another example, the media comprises 5% (v/v) newborn mammalian serum. In another example, the media comprises 5% (v/v) newborn mammalian serum and 5% (v/v) fetal bovine serum. In another example, the media is serum free.

In an example, the MLPSCs in the composition express an increased level of angiogenic marker(s) relative to a control population. In an example, the MLPSCs in the composition express an increased level of angiogenin relative to a control population. In an example, conditioned media from the MLPSCs induce increased levels of one or more of endothelial network formation, endothelial length, or endothelial branch length, relative to a control population. In an example, the control population is a population of MLPSCs that have been culture expanded in a cell culture medium comprising 10% fetal serum.

In an example, the the MLPSCs express a level of angiogenin greater than about 1200 pg/ml. In another example, the MLPSCs express a level of SDF-1 greater than about 3000 pg/ml. In another example, the MLPSCs express a level of VEGF greater than about 3200 pg/ml.

In an example, conditioned media from the MLPSCs induces endothelial network formation greater than about 0.12 mm²/mm². In an example, conditioned media from the MLPSCs induces endothelial network length greater than about 5 mm²/mm². In an example, conditioned media from the MLPSCs induces endothelial branch length greater than about 15 1/mm².

In a further aspect, the present disclosure relates to an in vitro method for pre-licensing MLPSCs, the method comprising culturing a human cell population enriched for MLPSCs in a serum-containing cell culture medium suitable for maintenance and proliferation of MLPSCs; wherein the serum is a serum comprising one or more pro-inflammatory cytokines. In an example, the disclosure relates to a composition produced by the aforementioned example method.

In another aspect, the present disclosure relates to a cryopreserved composition comprising:
(i) a culture-expanded population of mesenchymal lineage precursor or stem cells (MLPSCs), wherein the MLPSCs have been culture expanded in newborn mammalian serum obtained no more than 21 days after birth; and
(ii) a cryopreservative.

In some embodiments of any of the foregoing compositions, culture media, or methods the one or more pro-inflammatory cytokines comprise: IL-1β, IL-6, IFN-γ, TNF-α, or IL-1 receptor antagonist (IL-1RA). In some embodiments the one or more pro-inflammatory cytokines comprise: IL-1β, IL-6, IFN-γ, TNF-α, and IL-1RA.

In some embodiments of any of the foregoing compositions, culture media, or methods the serum comprising the one or more pro-inflammatory cytokines comprises newborn serum. In some preferred embodiments the newborn serum is bovine newborn serum. In some embodiments, the newborn serum is postnatal day 1 to postnatal day 7. In some embodiments the newborn serum is postnatal day 1 newborn serum. In some embodiments the newborn serum is postnatal day 1 to postnatal day 20.

In some embodiments of any of the foregoing compositions, culture media, or methods, where the serum comprising one or more inflammatory cytokines is newborn serum, the newborn serum is at a concentration of about 2% (v/v) to about 12% (v/v). In some preferred embodiments the concentration of newborn serum is about 5% (v/v). In other embodiments the concentration of newborn serum is about 10% (v/v).

In some embodiments the concentration of the cytokines in the newborn serum is:
(i) about 2 ng/ml to about 50 ng/ml for IL-1;
(ii) about 2 ng/ml to about 50 ng/ml for IL-1β;
(iii) about 0.2 ng/ml to about 1.2 ng/ml for IL-6;
(iv) about 0.1 ng/ml to about 0.2 ng/ml for IFN-γ;
(v) about 6 ng/ml to about 33 ng/ml for IL-1ra; or
(vi) about 0.1 ng/ml to about 0.7 ng/ml for TNF-α.

In some embodiments the concentration of the cytokines in the newborn serum is:
(i) about 2 ng/ml to about 50 ng/ml for IL-1;
(ii) about 2 ng/ml to about 50 ng/ml for IL-1β;
(iii) about 0.2 ng/ml to about 1.2 ng/ml for IL-6;
(iv) about 0.1 ng/ml to about 0.2 ng/ml for IFN-γ;
(v) about 6 ng/ml to about 33 ng/ml for IL-1ra; and
(vi) about 0.2 ng/ml to about 0.7 ng/ml for TNF-α.

In some embodiments of any of the foregoing compositions, culture media, or methods, in addition to newborn serum, fetal serum is also included or used. In some embodiments, where both fetal and newborn serum are to be included or used, the ratio of the concentration of the fetal serum to the concentration of the newborn serum is 1:1. In some embodiments, where the ratio of fetal to newborn serum concentration is 1:1, the concentration of the fetal serum and the concentration of newborn serum are each 5% (v/v). In other embodiments the concentration of the fetal serum is lower than the concentration of newborn serum.

In some embodiments of any of the foregoing compositions, culture media, or methods the newborn serum is substantially the sole source of exogenous pro-inflammatory cytokines.

In some embodiments of any of the foregoing methods, the method also includes a step of determining or having determined the level of the one or more pro-inflammatory cytokines in the serum.

In some embodiments of any of the foregoing compositions, culture media, or methods, the MLPSCs are maintained in an undifferentiated state.

In some embodiments of any of the foregoing compositions, culture media, or methods, the MLPSCs are human mesenchymal stem cells (hMSCs). In other embodiments, the MLPSCs are culture expanded from a population of STRO-1⁺ multipotential cells.

In another aspect, the present disclosure relates to a media for culturing MLPSCs, the media comprising:
IFN-gamma and/or TNF-alpha; and/or,
one or more pro-inflammatory cytokines, wherein the pro-inflammatory cytokines are selected from the group consisting of IL-6; IL-8; IL-17A; MCP-1; MIP-1-alpha; MIP-1-beta; IP-10.

For example, the media may comprise IFN-gamma and/or TNF-alpha. In an example, the level(s) of IFN-gamma and/or TNF-alpha in the media are <1 ng.

In an example, the media contains three or more pro-inflammatory cytokines. In an example, the media contains two or more pro-inflammatory cytokines selected from the group consisting of IL-6; IL-8; IL-17A; MCP-1; MIP-1-alpha; MIP-1-beta; IP-10. In an example, the media contains IL-6. In another example, the media contains IL-8 and/or IL-17A. In an example, the media contains IFN-gamma and TNF-alpha. In an example, the level of IFN-gamma is <500 pg/ml. In an example, the level of IFN-gamma is <100 pg/ml. In another example, the level of TNF-alpha is <750 pg/ml. In an example, the level of TNF-alpha is <400 pg/ml.

In an example, the media contains serum which comprises the pro-inflammatory cytokines. In an example, the serum is a newborn mammalian serum such as, for example, newborn calf serum. In an example, the newborn serum is obtained no more than 21 days after birth.

In an example, the media is characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

In an example, the media is characterised by a level of IFN-gamma greater than 1 pg/ml; a level of TNF-alpha greater than 2 pg/ml; and, one or more or all of the following:
i. a level of IL-6 greater than 3 pg/ml;
ii. a level of IL-8 greater than 500 pg/ml;
iii. a level of IL-17A greater than 0.2 pg/ml;
iv. a level of MCP-1 greater than 3 pg/ml;
v. a level of MIP-1-alpha greater than 0.5 pg/ml;
vi. a level of MIP-1-beta greater than 3 pg/ml;
vii. a level of IP-10 greater than 500 pg/ml

In an example, the media comprises at least 5% (v/v) newborn mammalian serum. In another example, the media comprises 5% (v/v) newborn mammalian serum.

In an example, the media is serum free. For example, the present disclosure encompasses a serum free MLPSC culture medium, which comprises:
IFN-gamma and/or TNF-alpha; and,
one or more pro-inflammatory cytokines, wherein the pro-inflammatory cytokines are selected from the group consisting of IL-6; IL-8; IL-17A; MCP-1; MIP-1-alpha; MIP-1-beta; IP-10.

### BRIEF DESCRIPTION OF DRAWINGS

FIG. 1: Serum cytokine levels assessment and comparison. 1:1 FCS/NBCS (serum A); fetal bovine serum (serum B); FBS from a different supplier (serum C).

FIG. 2: Quantitative measurement of in-vitro angiogenesis induced by MLPSC conditioned media using IncuCyte® 96-Well Kinetic Angiogenesis PrimeKit Assay.

FIG. 3: Luminex assay results showing increased production of angiogenin by MLPSCs cultured in the presence or absence of newborn serum.

FIG. 4: Angiogenic marker levels in MLPSC-conditioned media from cGMP lots cultured in the presence or absence of newborn serum.

FIG. 5: Analysis of Change from Baseline at 12 Months in Echo Parameters—all subjects.

FIG. 6: Analysis of Change from Baseline at 12 Months in Echo Parameters—subjects with persistent inflammation (hsCRP ≥2).

FIG. 7: Analysis of Change from Baseline at 12 Months in Echo Parameters—subjects without persistent inflammation (hsCRP <2).

FIG. 8: CV Death in in subjects with persistent inflammation (hsCRP ≥2) by MPCs cultured in the presence or absence of newborn serum.

FIG. 9: 3-Point composite MACE (MI, Stroke or CV Death) in subjects with persistent inflammation (hsCR ≥2) by MPCs cultured in the presence or absence of newborn serum.

FIG. 10: 3-Point composite MACE (MI, Stroke or CV Death) in subjects administered MPCs cultured in the presence or absence of newborn serum in all or final passages. LHS figure represents data from all patients. RHS figure represents data from patients with persistent inflammation (CRP >2 mg/ml).

FIG. 11: 3-Point composite MACE (LHS figure) and Terminal Cardiac Events (TCE; RHS figure) in subjects with most severe disease (NTpro-BNP >1000 ng/ml; CRP >2 mg/ml) administered MPCs cultured in the presence or absence of newborn serum in all or final passages.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular biology, stem cell biology, and biochemistry).

Unless otherwise indicated, cell culture techniques and assays utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

“C-reactive protein” or “CRP” is an inflammatory mediator. CRP levels are raised under conditions of acute inflammatory recurrence and rapidly normalize once the inflammation subsides. Accordingly, CRP is an effective marker of persistent inflammation. In an example, the term “persistent inflammation” is used to refer to subjects that have elevated CRP. The term “elevated CRP” is used in the context of the present disclosure to refer to CRP levels that are increased relative to baseline CRP levels. In an example, CRP levels ≥1 mg/L are elevated. In another example, CRP levels ≥1.5 mg/L are elevated. In another example, CRP levels ≥2 mg/L are elevated. Accordingly, in an example, persistent inflammation is characterised by CRP levels ≥2 mg/L.

N-terminal pro-B-type natriuretic peptide (NT-proBNP) is an inactive peptide released along with the active peptide hormone BNP when the walls of the heart are stretched or there is pressure overload on the heart. In the context of the present disclosure, high risk patients (e.g. patients at high risk of cardiovascular death) may be defined based on their level of NT-proBNP. In an example, the level is >1000 pg/mL, or, between 1000 pg/ml and 2500 pg/ml. In an example, these patients also have persistent inflammation. For example, these patients can have CRP levels ≥2 mg/L.

The term “level” is used to define the amount of a particular substance present in a sample, cell culture medium, serum preparation or compositions of the present disclosure. For example, a particular concentration, weight, percentage (e.g. v/v %) or ratio can be used to define the level of a particular substance.

In an example, the level of a particular marker is determined under culture conditions. The term “culture conditions” is used to refer to cells growing in culture. In an example, culture conditions refers to an actively dividing population of cells. Such cells may, in an example, be in exponential growth phase.

In an example, culture conditions encompass co-culture of an MLPSC population disclosed herein and a second cell population such as a population which comprises peripheral blood mononuclear cells (PBMC). In an example, co-culture comprises culturing an MLPSC population disclosed herein and a population of activated PBMC. For example, PBMC can be activated using anti-CD3 and anti-CD28 antibodies before co-culture with an MLPSC population disclosed herein.

In an example, “culture conditions” comprises co-culturing MLPSCs and T cells at a ratio of about 1 MLPSC:2 T cells, or less. For example, 1:3, 1:4, 1:5, 1:10, 1:20, 1:30, 1:40, 1:50, 1:60, 1:70 1:80, 1:90, or 1 MLPSC:100 T cells, or less. In this example, the level of IL2-RA inhibition is determined after about 30 to 84 hours of cell culture under culture conditions.

In an example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art will appreciate that secreted markers can be measured by sampling the culture media while markers expressed on the surface of the cell may be measured by assessing a sample of cell lysate. In an example, the sample is taken when the cells are in exponential growth phase. In an example, the sample is taken after at least two or three days in culture. In another example, the sample is taken after about 30 to 84 hours of culture. In an example, the sample is taken from a co-culture of MLPSCs and activated PBMCs. In this example, the cell sample can be lysed and the level of a marker can be determined. For example, the level of IL2-RA may be determined. In his example, the level of IL2-RA can be determined using various methods such as an enzyme-linked immunosorbent assay (ELISA) based method. In an example, the ELISA comprises:
(i) adding sample diluent to each well of a microplate precoated with a monoclonal antibody specific for IL2-RA;
(ii) adding a co-cultured sample to a well of a microplate precoated with a monoclonal antibody specific for IL2-RA;
(iii) incubating the microplate for sufficient time to allow for the monoclonal antibody specific for IL2-RA to specifically bind to any IL2-RA in the sample;
(iv) washing the microplate;
(v) adding IL2-RA conjugate to the well;
(vi) incubating the microplate for sufficient time to allow the conjugate to specifically bind to any captured IL2-RA;
(vii) washing the microplate;
(viii) adding a substrate solution to the well;
(ix) incubating the microplate for sufficient time for colour development;
(x) adding a stop solution to the well;
(xi) reading optical density on a microplate reader set to 450 nm with wavelength correction at 570 nm;
(xii) determining the level of IL2-RA.

In another example, the level of IL2-RA is determined using fluorescence-activated cell sorting (FACS) using appropriate antibodies such as anti-CD25. Further antibodies may also be employed if required to distinguish CD25+ cell types. While the above referenced examples refer to IL-2RA, it will be appreciated that similar methods may also be used to determine the level of other markers disclosed herein such as angiogenin. In these examples, co-culture may not be required to determine the level. For example, the level of angiogenin may be measured in a population of MLPSCs under culture conditions.

In another example, the level is measured based on an assessment of conditioned media (or properties thereof) obtained from a population of MLPSCs under culture conditions. For example, conditioned media can be obtained from a population of MLPSCs disclosed herein under culture conditions before being used in one or more angiogenesis assays disclosed below.

In the context of the present disclosure, the term “sufficient” is used to define an amount that provides a specific concentration when dissolved in a stem cell culture medium. A “sufficient amount” is dictated by the volume of culture medium required.

The term “angiogenic marker” as used herein refers an indicator of angiogenesis. As used herein, “angiogenic markers” include pro-angiogenic molecules, for example, VEGF, angiogenin, and SDF-1α. In another example, angiogenic markers are cellular indicators of angiogenesis, for example, endothelial network formation, endothelial network length, and endothelial branch length. In this example, cellular indicators of angiogenesis are determined in an in-vitro angiogenesis assay as disclosed herein. In an example, angiogenic marker characterisation may be used to characterise a MLPSC population disclosed herein (e.g. a cryopreserved intermediate or drug product disclosed herein).

In an example, compositions of the disclosure comprise genetically unmodified MLPSCs. As used herein, the term “genetically unmodified” refers to cells that have not been modified by transfection with a nucleic acid. For the avoidance of doubt, in the context of the present disclosure a MLPSC transfected with a nucleic acid encoding a protein would be considered genetically modified.

As used herein, the term “sample” refers to an extract from a cell culture in which the level of a particular marker can be measured. The “sample” includes extracts and/or derivatives and/or fractions of the sample. In an example, the “sample” is a population of cells, for example a population of cells under culture conditions. In an example, the sample is supernatant obtained following cell culture, for example, cell conditioned media. In these examples, the sample is any extract of cell culture in which angiogenic markers can be measured. In an example, the sample is contacted with another cell population to determine the level of an angiogenic marker. In another example, the sample is obtained from a patient (e.g. blood sample). Such samples can be used to determine the level of markers such as CRP.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

### Mesenchymal Lineage Precursor or Stem Cells (MLPSCs)

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+ MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+). For example, the MPCs can be isolated from bone mononuclear cells with an anti-STRO-3 antibody.

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+(HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

The term “pre-licensing” or “licensing”, as used herein, refers to a process by which MLPSCs achieve functional maturation, whereby, the pre-licensed or licensed MLPSCs reduce release of inflammatory cytokines when the MLPSCs are administered to a subject to a greater extent than MLPSCs that have not been pre-licensed.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for STRO-1+ cells comprises at least about 0.1% or 0.5% or 1% or 2% or 5% or 10% or 15% or 20% or 25% or 30% or 50% or 75% STRO-1+ cells. In this regard, the term “population of cells enriched for STRO-1+ cells” will be taken to provide explicit support for the term “population of cells comprising X % STRO-1+ cells”, wherein X % is a percentage as recited herein. The STRO-1+ cells can, in some examples, form clonogenic colonies, e.g. CFU-F (fibroblasts) or a subset thereof (e.g., 50% or 60% or 70% or 80% or 90% or 95%) can have this activity.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1^{bright}). Accordingly, an indication that cells are STRO-1+ does not mean that the cells are selected by STRO-1 expression. In one example, the cells are selected based on at least STRO-3 expression, e.g., they are STRO-3+ (TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the mesenchymal lineage precursor or stem cells used in the present disclosure express one or more markers individually or collectively selected from the group consisting of TNAP+, VCAM-1+, THY-1+, STRO-2+, STRO-4+ (HSP-90β), CD45+, CD146+, 3G5+ or any combination thereof.

By use of the term “individually” it is meant that the disclosure encompasses the recited markers or groups of markers separately, and that, notwithstanding that individual markers or groups of markers may not be separately listed herein the accompanying claims may define such marker or groups of markers separately and divisibly from each other.

By use of the term “collectively” it is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

In one example, the STRO-1+ cells are STRO-1^{bright} (syn. STRO-1^{bri}). In another example, the STRO-1^{bri} cells are preferentially enriched relative to STRO-1^{dim} or STRO-1^{intermediate} cells. In another example, the STRO-1^{bri} cells are additionally one or more of TNAP+, VCAM-1+, THY-1+, STRO-2+, STRO-4+ (HSP-90β) and/or CD146+. For example, the cells are selected for one or more of the foregoing markers and/or shown to express one or more of the foregoing markers. In this regard, a cell shown to express a marker need not be specifically tested, rather previously enriched or isolated cells can be tested and subsequently used, isolated or enriched cells can be reasonably assumed to also express the same marker.

In one example, the mesenchymal precursor cells are perivascular mesenchymal precursor cells as defined in WO 2004/85630, characterized by the presence of the perivascular marker 3G5.

A cell that is referred to as being “positive” for a given marker may express either a low (lo or dim) or a high (bright, bri) level of that marker depending on the degree to which the marker is present on the cell surface, where the terms relate to intensity of fluorescence or other marker used in the sorting process of the cells. The distinction of lo (or dim or dull) and bri will be understood in the context of the marker used on a particular cell population being sorted. A cell that is referred to as being “negative” for a given marker is not necessarily completely absent from that cell. This term means that the marker is expressed at a relatively very low level by that cell, and that it generates a very low signal when detectably labelled or is undetectable above background levels, e.g., levels detected using an isotype control antibody.

The term “bright” or “bri” as used herein, refers to a marker on a cell surface that generates a relatively high signal when detectably labelled. Whilst not wishing to be limited by theory, it is proposed that “bright” cells express more of the target marker protein (for example the antigen recognized by STRO-1) than other cells in the sample. For instance, STRO-1^{bri} cells produce a greater fluorescent signal, when labelled with a FITC-conjugated STRO-1 antibody as determined by fluorescence activated cell sorting (FACS) analysis, than non-bright cells (STRO-^{dull/dim}). In one example, “bright” cells constitute at least about 0.1% of the most brightly labelled bone marrow mononuclear cells contained in the starting sample. In other examples, “bright” cells constitute at least about 0.5%, at least about 1%, at least about 1.5%, or at least about 2%, of the most brightly labelled bone marrow mononuclear cells contained in the starting sample. In an example, STRO-1^{bright} cells have 2 log magnitude higher expression of STRO-1 surface expression relative to “background”, namely cells that are STRO-1⁻. By comparison, STRO-1^{dim} and/or STRO-1^{intermediate} cells have less than 2 log magnitude higher expression of STRO-1 surface expression, typically about 1 log or less than “background”.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ multipotential cells are capable of differentiation into at least two different germ lines. Non-limiting examples of the lineages to which the multipotential cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an aspect of the present disclosure, the presently described mesenchymal lineage precursor or stem cells are MSCs. The MSCs may be a homogeneous composition or may be a mixed cell population enriched in MSCs. Homogeneous MSCs cell compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium.

In another example, the mesenchymal lineage precursor or stem cells are CD29+, CD54+, CD73+, CD90+, CD102+, CD105+, CD106+, CD166+, MHC1+ MSCs (e.g. remestemcel-L).

As will be appreciated by those of skill in the art, cultured mesenchymal lineage precursor or stem cells are phenotypically different to cells in-vivo. For example, in one embodiment they express one or more of the following markers, CD44, NG2, DC146 and CD140b. Cultured mesenchymal lineage precursor or stem cells are also biologically different to cells in-vivo, having a higher rate of proliferation compared to the largely non-cycling (quiescent) cells in-vivo.

Mesenchymal lineage precursor or stem cells cultured using the methods of the present disclosure may also be cryopreserved.

### Culture Expanded MLPCs

In example, culture expanded MLPSCs of the disclosure are characterised by expression of an angiogenic marker(s). For example, a culture expanded MLPSC population according to the present disclosure can be characterised by increased levels of VEGF, angiogenin, and/or SDF-1α under culture conditions. In another example, the MLPSC population can be characterised based on an assessment of conditioned media obtained from the MLPSC population under culture conditions. In an example, the conditioned media increases the level endothelial network formation, endothelial network length, and/or endothelial branch length in a population of endothelial cells when said cells are treated with conditioned media obtained from culture expanded MLPSCs. In an example, the increase is determined relative to a control population of MLPSCs. In an example, the control population is a population of MLPSCs that have been culture expanded in a cell culture medium comprising 10% fetal serum.

In an example, the expanded MLPSC population is characterised by a level of VEGF greater than about 3 ng/ml. In an example, the level of VEGF is between about 3 ng/ml to 4 ng/ml. In an example, the level of VEGF is greater than about 3.1 ng/ml. In an example, level of VEGF is greater than about 3.2 ng/ml. In an example, level of VEGF is greater than about 3.3 ng/ml. In an example, the level of VEGF is greater than about 3.4 ng/ml. In an example, the level of VEGF is greater than about 3.5 ng/ml. In an example, the level of VEGF is between about 3.2 and 3.6 ng/mL. In an example, the level of VEGF is about 3.45 ng/mL.

In an example, the MLPSCs express an increased level of angiogenin relative to a control population. In an example, the expanded MLPSC population is characterised by a level of angiogenin greater than about 1000 pg/ml. In an example, the level of angiogenin is greater than about 1100 pg/ml. In an example, the level of angiogenin is between about 1000 pg/ml and 1200 pg/ml. In an example, the level of angiogenin is between about 1100 pg/ml and 1150 pg/ml. In an example, the level of angiogenin is about 1114 pg/ml.

In an example, the expanded MLPSC population is characterised by a level of SDF-1α greater than about 3000 ng/ml. In an example, the level of SDF-1α is greater than about 3100 ng/ml. In an example, the level of SDF-1α is greater than about 3200 ng/ml. In an example, the level of SDF-1α is greater than about 3300 ng/ml. In an example, the level of SDF-1α is greater than about 3400 ng/ml. In an example, the level of SDF-1α is greater than about 3500 ng/ml. In an example, the level of SDF-1α is between about 3000 ng/ml and 3500 ng/ml. In an example, the level of SDF-1α is between about 3000 ng/ml and 3400 ng/ml. In an example, the level of SDF-1α is between about 3000 ng/ml and 3300 ng/ml. In an example, the level of SDF-1α is between about 3100 ng/ml and 3400 ng/ml. In an example, the level of SDF-1α is between about 3100 ng/ml and 3300 ng/ml.

In an example, the culture expanded MLPSC population is characterised by conditioned media which stimulates endothelial network formation greater than about 0.1 mm²/mm². In an example, the endothelial network formation is between about 0.1 mm²/mm² and 0.2 mm²/mm². In another example, the endothelial network formation is about 0.12 mm²/mm².

In an example, the culture expanded MLPSC population is characterised by conditioned media which stimulates endothelial network length greater than about 4 mm²/mm². In an example, the endothelial network length is between about 4 mm²/mm² and about 6 mm²/mm². In an example, the endothelial network length is about 5 mm²/mm². In an example, the culture expanded MLPSC population is characterised by conditioned media which stimulates endothelial branch length greater than about 12 1/mm². In an example, the endothelial branch length is between about 12 1/mm² and about 17 1/mm². In an example, the endothelial branch length is about 15 1/mm².

In an example, the culture expanded MLPSC population is characterised by an increased level of one or more angiogenic markers relative to a population of MLPSCs that have been culture expanded in a cell culture medium comprising 10% fetal serum. In an example, the level of angiogenic marker is increased by about 5%, about 10%, about 20%, about 30%, about 40%, about 50%, about 60%, or about 70% relative to a population of MLPSCs that have been culture expanded in a cell culture medium comprising 10% fetal serum. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%. In an example, the level of angiogenic marker is increased relative to a population of MLPSCs that have been culture expanded in cell culture medium that does not contain IFN-gamma or TNF-alpha.

In an example, the expanded MLPSC population is characterised by an increased level of one or more angiogenic markers relative to a population of MLPSCs that have been culture expanded in a cell culture medium that does not comprise newborn serum. In an example, the level of angiogenic marker is increased by about 5%, about 10%, about 20%, about 30%, about 40%, about 50%, about 60%, or about 70% relative to a population of MLPSCs that have been culture expanded in a cell culture medium that does not comprise newborn serum. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%.

In another example, culture expanded MLPSCs of the disclosure are characterised based on therapeutic efficacy. For example, the MLPSCs may be characterised based on therapeutic efficacy in an inflammatory disease. In an example, the MLPSCs are characterised by therapeutic efficacy in heart failure. In another example, the MLPSCs are characterised by therapeutic efficacy in a T-cell mediated disease such as GvHD.

In another example, culture expanded MLPSCs are characterised by their capacity to inhibit IL-2RA expression by CD3/CD28-activated PBMCs under culture conditions. In an example, the culture expanded MLPSCs inhibit IL2-RA expression by CD3/CD28-activated PBMCs by at least 60% relative to a control. In another example, the culture expanded MLPSCs inhibit IL2-RA expression by CD3/CD28-activated PBMCs by at least 65% relative to a control. In another example, the culture expanded MLPSCs inhibit IL2-RA expression by CD3/CD28-activated PBMCs by at least 70% relative to a control. In another example the culture expanded MLPSCs inhibit IL2-RA expression by CD3/CD28-activated PBMCs by between 60 and 70% relative to a control.

“Culture expanded” MLPSCs are distinguished from freshly isolated cells in that they have been cultured in cell culture medium and passaged (i.e. sub-cultured).

In an example, freshly isolated cells are culture expanded for about 1 or 2 passages to provide an intermediate population. In an example, freshly isolated cells are culture expanded for 2 passages to provide an intermediate population. In another example, freshly isolated cells are culture expanded for about 1 to 3 passages to provide an intermediate population. In an example, freshly isolated cells are STRO-1+.

Accordingly, in an example, relevant cells are isolated and culture expanded for 2 passages to provide an intermediate MLPSC population. In certain examples, the intermediate MLPSC population is then culture expanded to provide a drug product (DP). For example, DP compositions of the present disclosure are produced by culturing cells from an intermediate cryopreserved MLPSC population or, put another way, a cryopreserved intermediate. In an example, the intermediate cell population can be cultured for three more passages (i.e. 5 passages total) to provide a DP.

In an example, MLPSCs are culture expanded for about 4-10 passages. In an example, MLPSCs are culture expanded for at least 5, at least 6, at least 7, at least 8, at least 9, at least 10 passages. For example, MLPSCs can be culture expanded for at least 5 passages. In an example, MLPSCs can be culture expanded for at least 5-10 passages. In an example, MLPSCs can be culture expanded for at least 5-8 passages. In an example, MLPSCs can be culture expanded for at least 5-7 passages. In an example, MLPSCs can be culture expanded for more than 7 passages. In these examples, MLPSCs may be culture expanded before being cryopreserved to provide an intermediate cryopreserved MLPSC population and then subject to further culture expansion.

In an example, compositions of the disclosure comprise MLPSCs that are culture expanded from a cryopreserved intermediate. In an example, the cells culture expanded from a cryopreserved intermediate are culture expanded for at least 3, at least 5, at least 6, at least 7, at least 8, at least 9, at least 10 passages. For example, MLPSCs can be culture expanded for at least 3 passages. In an example, MLPSCs can be culture expanded for at least 3-10 passages. In an example, MLPSCs can be culture expanded for at least 3-8 passages. In an example, MLPSCs can be culture expanded for at least 3-7 passages. In an example, MLPSCs culture expanded from a cryopreserved intermediate are culture expanded in media disclosed herein (e.g. media containing newborn calf serum).

In an example, MLPSCs can be obtained from a single donor, or multiple donors where the donor samples or MLPSCs are subsequently pooled and then culture expanded as required. In an example, the culture expansion process comprises:
i. expanding by passage expansion the number of viable cells to provide a preparation of at least about 1 billion of the viable cells, wherein the passage expansion comprises establishing a primary culture of isolated MLPSCs and then serially establishing a first non-primary (P1) culture of isolated MLPSCs from the previous culture;
ii. expanding by passage expansion the P1 culture of isolated MLPSCs to a second non-primary (P2) culture of MLPSCs; and,
iii. preparing and cryopreserving an in-process intermediate MLPSC preparation obtained from the P2 culture of MLPSCs; and, optionally
iv. thawing the cryopreserved in-process intermediate MLPSC preparation and expanding by passage expansion the in-process intermediate MLPSC preparation.

In an example, the methods of the disclosure comprise selecting an intermediate population (e.g. a cryopreserved intermediate) for further culture expansion based on certain criteria such as the level of one more angiogenic markers. Selection processes are not particularly limited so long as they are able to select cell populations characterized by the relevant criteria such as level of angiogenic marker. In an example, a series of intermediate MLPSC populations are assessed for levels of angiogenic markers and those populations which express over a threshold level of the angiogenic marker as described herein are selected for further expansion.

It should be appreciated that the selection process does not require immediate culture expansion. Rather “selected” populations can be cryopreserved and culture expanded at a later stage. In an example, a fraction of the intermediate cell population is culture expanded with the remainder of the population being cryopreserved for culture expansion at a later stage.

In an example, selected cell populations are immediately culture expanded. In another example, selected cell populations are cryopreserved to allow culture expansion at a later stage.

In an example, a selected cell population is culture expanded to provide a pharmaceutical composition. In an example, the pharmaceutical composition is characterized by certain criteria such as level of angiogenic markers.

In the context of the present disclosure, the level of angiogenic marker/s can be assessed between steps iii and iv of the culture expansion process described above. For example, the level of angiogenic marker/s may be determined under culture conditions and/or from conditioned media after step iii. In an example, step iv is only performed if a desired level of angiogenic marker/s is/are observed under culture conditions and/or from conditioned media. In this example, the cell population is selected for culture expansion on the basis of the level of angiogenic marker/s under culture conditions and/or from conditioned media.

In an example, the culture expanded MLPSC population is expanded from an intermediate MLPSC population with an increased level of one or more angiogenic markers relative to a population of MLPSCs that have been culture expanded in a cell culture medium comprising 10% fetal serum.

In an example, a level of an angiogenic marker(s) disclosed herein is considered increased when it is increased by about 5%, about 10%, about 20%, about 30%, about 40%, about 50%, about 60 %, or about 70% relative to a population of MLPSCs that have been culture expanded in a cell culture medium comprising 10% fetal serum. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%.

In an example, the culture expanded MLPSC population is expanded from an intermediate MLPSC population with an increased level of one or more angiogenic markers relative to a population of MLPSCs that have been culture expanded in a cell culture medium that does not comprise newborn serum. In an example, the level of angiogenic marker is considered increased when it is increased by about 5%, about 10%, about 20%, about 30%, about 40%, about 50%, about 60 %, or about 70% relative to a population of MLPSCs that have been culture expanded in a cell culture medium that does not comprise newborn serum. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%.

In an example, the culture expanded MLPSC preparation has an antigen profile and an activity profile comprising:
i. less than about 0.75% CD45+ cells;
ii. at least about 95% CD105+ cells;
iii. at least about 95% CD166+ cells.

The process of MLPSC isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although MLPSCs can generally withstand a certain amount of insult during preparation, cells are preferably manipulated by handling procedures and/or equipment that adequately performs the given step(s) while minimizing insult to the cells.

In an example, MLPSCs are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a MLPSC composition cultured according to the present disclosure is 95% homogeneous with respect to being CD105 positive and CD166 positive and being CD45 negative. In an example, this homogeneity persists through ex vivo expansion; i.e. though multiple population doublings.

In an example, MLPSCs of the disclosure are culture expanded in 2D culture. For example, MLPSCs of the disclosure can be culture expanded in a cell factory. In certain examples, 3D culture of intermediates disclosed herein may follow using, for example, a bioreactor. In an example, MLPSCs of the disclosure are initially culture expanded in 2D culture prior to being further expanded in 3D culture. In an example, intermediate cell populations of the disclosure have not been culture expanded in 3D culture. In an example, the level of one or more angiogenic markers is assessed before subsequent culture expansion in a cell factory or 3D culture.

In an example, MLPSCs of the disclosure are culture expanded from an intermediate population. In an example, MLPSCs of the disclosure are culture expanded from the intermediate in 2D culture before seeding in 3D culture.

In the context of both intermediate populations and therapeutic compositions expanded from the same, in an example, MLPSCs of the disclosure are culture expanded in 2D culture for at least 3 days before seeding in a further culture system such as cell factory or 3D culture in a bioreactor. In an example, MLPSCs of the disclosure are culture expanded in 2D culture for at least 4 days before seeding in a further culture system. In an example, MLPSCs of the disclosure are culture expanded in 2D culture for between 3 and 5 days before seeding in a further culture system. In these examples, 2D culture can be performed in a cell factory. Various cell factory products are available commercially (e.g. Thermofisher, Sigma, Corning). In an example, the cell factory has at least 5 layers. In an example, the cell factory has at least 10 layers. In an example, the cell factory has at least 20 layers. 3D culture may be performed in various bioreactor types such as stirred tank, wave bag, and vertical wheel.

In an example, CO₂ is provided during culture expansion of MLPSCs. In an example, MLPSCs are culture expanded in less than 9% CO₂. In an example, MLPSCs are culture expanded in less than 8% CO₂. In an example, MLPSCs are culture expanded in 5% CO₂. For example, MLPSCs can be culture expanded in 5% +/−2% CO₂. In an example, the MLPSCs are culture expanded with passive priming of CO₂. For example, cell factories can be passively primed with 5% CO₂.

Priming cell factories maintains the CO₂ tension between the cell factory and incubator and stabilizes the pH level of the growth medium. Active priming involves actively passing CO₂ gas through a bacterial vent air filter into each culture vessel (e.g. cell factory) for a defined period of time (e.g. around 10 minutes). However, active priming has the potential to introduce contamination into culture as it requires an open port to provide gas. Passive priming involves placing a closed culture system into an incubator at appropriate CO₂ concentration prior to cell seeding (e.g. around 12 to 72 hours). In an example, cells of the disclosure are STRO-3+ before they are culture expanded to provide an intermediate cell population.

### Compositions

The compositions disclosed herein are useful for pre-licensing of MLPSCs, a multistep process that leads to the functional maturation of MSCs that promotes their therapeutic potency.

Accordingly provided herein is a composition for pre-licensing MLPSCs that contains a human cell population enriched for MLPSCs; and a serum, where the serum is a serum containing one or more pro-inflammatory cytokines. In other embodiments disclosed herein the composition includes (i) a human cell population enriched for MLPSCs; (ii) serum comprising one or more pro-inflammatory cytokines; and (iii) a cryopreservative. Suitable cryopreservatives include, but are not limited to one or more of: dimethylsulfoxide (DMSO), trehalose, and albumin.

In other embodiments provided herein is a cell culture medium for proliferation and pre-licensing of MLPSCs, where the cell culture medium includes a serum containing one or more pro-inflammatory cytokines.

The cell culture medium of the present disclosure can contain any components such as fatty acids or lipids, vitamins, cytokines, antioxidants, buffering agents, inorganic salts and the like.

The cell culture media used in the present disclosure contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest with key nutrients available at adequate levels to enhance cell proliferation. For example, it may be necessary to increase the level of glucose (or other energy source) in the basal medium, or to add glucose (or other energy source) during the course of culture, if this energy source is found to be depleted and to thus limit cell proliferation.

The culture media of the present disclosure can be prepared by using a basal culture medium. In the context of the present disclosure, “basal culture medium” refers to an unsupplemented medium which is suitable for exposure to cells, for example MSC. Basal culture medium includes, for example, Eagles minimal essential (MEM) culture media, alpha modified MEM culture media, StemSpan™ and mixed culture media thereof, and is not particularly restricted providing it can be used for culturing of MLPSCs.

In some preferred embodiments of the compositions for pre-licensing disclosed herein, the composition includes one or more pro-inflammatory cytokines selected from among IL-1β, IL-6, IFN-γ, TNF-α, and IL-1 receptor antagonist (IL-1ra). In some embodiments the composition includes each of IL-1β, IL-6, IFN-γ, TNF-α, and IL-1 receptor antagonist (IL-1ra). In some embodiments the composition is substantially free of pro-inflammatory cytokines other than IL-1β, IL-6, IFN-γ, TNF-α, and IL-1ra. In some embodiments the composition contains only 1, 2, 3, 4, or 5 pro-inflammatory cytokines, where the 1 to 5 pro-inflammatory cytokines are selected from the group consisting of IL-1β, IL-6, IFN-γ, TNF-α, and IL-1ra.

In some embodiments the concentration of IL-1β in a newborn serum to be included in the compositions disclosed herein is about 2 ng/ml to about 50 ng/ml, e.g., 5 ng/ml, 10 ng/ml, 15 ng/ml, 20 ng/ml, 25 ng/ml, 30 ng/ml, 40 ng/ml, 45 ng/ml, or another concentration from about 2 ng/ml to about 50 ng/ml. In some embodiments the concentration of IL-6 in a suitable newborn serum is about 0.2 ng/ml to about 1.2 ng/ml, e.g., 0.4 ng/ml, 0.5 ng/ml, 0.6 ng/ml, 0.7 ng/ml, 0.8 ng/ml, 1.0 ng/ml, or another concentration from about 0.2 ng/ml to about 1.2 ng/ml. In some embodiments the concentration of IFN-γ in a suitable newborn serum is about 0.1 ng/ml to about 0.2 ng/ml, e.g., 0.12 ng/ml, 0.14 ng/ml, 0.16 ng/ml, 0.18 ng/ml, or another concentration from about 0.1 ng/ml to about 0.2 ng/ml. In some embodiments the concentration of IL-1ra in a suitable newborn serum is about 6 ng/ml to about 33 ng/ml, e.g., 7 ng/ml, 8 ng/ml, 9 ng/ml, 10 ng/ml, 12 ng/ml, 15 ng/ml, 20 ng/ml, 22 ng/ml, 25 ng/ml, 27 ng/ml, 30 ng/ml, or another concentration from about 6 ng/ml to about 33 ng/ml. In some embodiments the concentration of TNF-α in a suitable newborn serum is about 0.1 ng to about 0.7 ng/ml, e.g., 0.2 ng/ml, 0.3 ng/ml, 0.4 ng/ml, 0.5 ng/ml, 0.6 ng/ml, or another concentration from about 0.2 ng/ml to about 0.7 ng/ml.

In some preferred embodiments, the compositions disclosed herein are substantially free of any pro-inflammatory cytokines other than the ones present in the serum prior to its inclusion in the composition, i.e., the serum to be used is substantially the sole source of exogenous pro-inflammatory cytokines in the composition.

In some embodiments the concentration of newborn serum in the compositions described is about 2% (v/v) to about 12% (v/v), e.g., 3%, 4%, 5%, 6%, 7%, 8%, 9%, 10%, 11%, 12%, or another concentration from about 2% (v/v) to about 12% (v/v). In some preferred embodiments the concentration of newborn serum is about 5% (v/v). In other preferred embodiments the concentration of newborn serum is about 10% (v/v).

In some embodiments the compositions disclosed herein contain both fetal serum and newborn serum. In some preferred embodiments the ratio of fetal serum to newborn serum in the composition is 1:1. In some embodiments the ratio of fetal serum to newborn serum is greater than 1:1, e.g., 3:1, 2.8:1, 2.5:1, 2.2:1, 2:1, 1.8:1, 1.5:1, 1.2:1, or another ratio of fetal serum to newborn serum of about 3:1 fetal serum to newborn serum or lower. In other embodiments, the ratio of fetal serum to newborn serum is lower than 1:1, e.g., 1:3, 1:2.8, 1:2.5, 1:2.2, 1:1.8, 1:1.5, 1:1.2, or another ratio of fetal serum to newborn serum of about 1:3 fetal serum to newborn serum or higher.

In some preferred embodiments, the total concentration of serum, including fetal and newborn serum at a given ratio, in a composition is about 10% (v/v).

In some embodiments, where the composition contains a cryopreservative, e.g., DMSO, the concentration of newborn serum is from about 3% (v/v) to about 20% (v/v), e.g., 4%, 6%, 7%, 7.5%, 8%, 9%, 10%, 12%, 15%, or another concentration from about 3% (v/v) to about 15% (v/v). In some embodiments, in a cryopreservation composition, the concentration of fetal and newborn serum is at a ratio of 1:1, e.g., 5% (v/v) of each.

In some embodiments the serum containing one or more pro-inflammatory cytokines to be used in the compositions disclosed herein is a newborn serum. In some preferred embodiments the newborn serum to be used is, e.g., newborn bovine calf serum, newborn lamb serum, and newborn equine foal serum. In some preferred embodiments the newborn serum is newborn bovine calf serum. In embodiments in which a composition disclosed herein contains newborn serum, the newborn serum is from a newborn at about postnatal day 1 to about postnatal day 7, e.g., postnatal day 2, postnatal day 3, postnatal day 4, postnatal day 5, postnatal day 6. For clarity postnatal day 1, as used herein, refers to the day of birth. In some embodiments, newborn serum used in the compositions described herein can contain a mixture of newborn sera obtained from different postnatal days.

In some embodiments the newborn serum to be used is bovine, ovine, caprine, equine, or human. In some preferred embodiments the newborn serum is bovine. In some embodiments, where a composition is to contain fetal serum, the fetal serum is bovine, ovine, equine, or caprine. In some preferred embodiments, the fetal serum is bovine fetal serum.

In an example, compositions of the disclosure comprise IFN-gamma and/or TNF-alpha (e.g. serum containing IFN-gamma and TNF-alpha). For example, the level of IFN-gamma can be less than 1 ng/ml. In an example the level of IFN-gamma is less than 500 pg/ml or less than 100 pg/ml. In an example, the level of TNF-alpha can be less than 1 ng/ml. In an example, the level of TNF-alpha is less than 750 pg/ml or less than 400 pg/ml. In an example, the composition comprises IFN-gamma and TNF-alpha and, the level of both is less than 1 ng/ml. In an embodiment of this example, the IFN-gamma and TNF-alpha are provided in serum.

In an example, the composition comprises one or more pro-inflammatory cytokines which are capable of binding a receptor on the surface of MLPSCs.

In an example, the serum comprises one or more pro-inflammatory cytokines selected from the group consisting of IL-6; IL-8; IL-17A; MCP-1; MIP-1-alpha; MIP-1-beta; IP-10. For example, the serum can comprise IL-8.

In an example, the composition comprises IFN-gamma and/or TNF-alpha, and, one or more pro-inflammatory cytokines selected from the group consisting of IL-6; IL-8; IL-17A; MCP-1; MIP-1-alpha; MIP-1-beta; IP-10. In an example, the level of IFN-gamma and/or TNF-alpha is less than 1 ng/ml.

In an example, the composition may comprise serum characterised by one or more or all of the following:
i. a level of IFN-gamma greater than 10 pg/ml;
ii. a level of TNF-alpha greater than 20 pg/ml;
iii. a level of IL-6 greater than 30 pg/ml;
iv. a level of IL-8 greater than 5,000 pg/ml;
v. a level of IL-17A greater than 2 pg/ml;
vi. a level of MCP-1 greater than 30 pg/ml;
vii. a level of MIP-1-alpha greater than 5 pg/ml;
viii. a level of MIP-1-beta greater than 30 pg/ml;
ix. a level of IP-10 greater than 5,000 pg/ml.

In certain examples, the serum may be diluted from neat concentration in cell culture media. In these examples, the levels of cytokines in the serum will be reduced accordingly. For example, the serum may be provided in the cell culture media at 10% (v/v). In this example, the serum may be characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

In an example, the serum is newborn serum, which comprises an above referenced level of cytokine(s). In an example, compositions of the disclosure comprise media described below.

In other examples, compositions of the disclosure comprises above referenced population(s) of culture expanded MLPSCs.

In some preferred embodiments the MLPSCs are human mesenchymal stem cells. In other embodiments the MLPSCs are STRO-1⁺ multipotential cells or a population of culture expanded MLPSCs that have been culture expanded from a STRO-1⁺ population of multipotential cells.

In preferred embodiments, the MLPSCs are maintained in an undifferentiated state.

### Media

In an embodiment the present disclosure encompasses MLPSC culture media supplemented with pro-inflammatory cytokine(s). In an example, the culture media comprises IFN-gamma and/or TNF-alpha. In an example, the media comprises IFN-gamma. For example, the level of IFN-gamma can be less than 1 ng/ml. In an example the level of IFN-gamma is less than 500 pg/ml or less than 100 pg/ml. In an example, the media comprises TNF-alpha. For example, the level of TNF-alpha can be less than 1 ng/ml. In an example, the level of TNF-alpha is less than 750 pg/ml or less than 400 pg/ml. In an example, the media comprises IFN-gamma and TNF-alpha and the level of both is less than 1 ng/ml.

In an example, the media comprises one or more pro-inflammatory cytokines which are capable of binding a receptor on the surface of MLPSCs.

In an example, the media comprises one or more pro-inflammatory cytokines selected from the group consisting of IL-6; IL-8; IL-17A; MCP-1; MIP-1-alpha; MIP-1-beta; IP-10. For example, the media can comprise IL-8.

In an example, the media comprises IFN-gamma and/or TNF-alpha, and, one or more pro-inflammatory cytokines selected from the group consisting of IL-6; IL-8; IL-17A; MCP-1; MIP-1-alpha; MIP-1-beta; IP-10. In an example, the level of IFN-gamma and/or TNF-alpha is less than 1 ng/ml.

In an example, the media is characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

In another example, the media comprises serum which is characterised by one or more or all of the following:
i. a level of IFN-gamma greater than 10 pg/ml;
ii. a level of TNF-alpha greater than 20 pg/ml;
iii. a level of IL-6 greater than 30 pg/ml;
iv. a level of IL-8 greater than 5,000 pg/ml;
v. a level of IL-17A greater than 2 pg/ml;
vi. a level of MCP-1 greater than 30 pg/ml;
vii. a level of MIP-1-alpha greater than 50 pg/ml;
viii. a level of MIP-1-beta greater than 30 pg/ml;
ix. a level of IP-10 greater than 5,000 pg/ml.

In an example, the media comprises IL-10. In another example, the media comprises IL-36RA. In another example, the media comprises IL-10 and IL-36RA. In an example, the level of IL-10 is greater than 0.3 pg/ml. For example, the level of IL-10 may be greater than 30 pg/ml. In an example, the level of IL-10 is greater than 400 pg/ml. In an example, the level of IL-36RA is greater than 50 pg/ml.

In an example, the media is serum free.

In an example, the media is serum free and supplemented with PDGF and FGF2. In an example, the medium is serum free and is supplemented with PDGF, FGF2 and EGF. In an example, the PDGF is PDGF-BB. In an example, the serum free media is supplemented with 10 ng/ml PDGF-BB, 5 ng/ml EGF and, 1 ng/ml FGF2.

In an example, the above referenced cytokines can be provided at a concentration <1 ng/ml each. For example, the media may be characterised by one or more or all of the following, each provided at <1ng/ml: IFN-gamma, TNF-alpha, IL-6, IL-17A, MCP-1, MIP-1-alpha, MIP-1-beta, IP-10.

### Methods

The present disclosure provides in vitro methods for pre-licensing of MLPSCs by culturing a human cell population enriched for MLPSCs (e.g., hMSCs) in a cell culture medium suitable for maintenance and proliferation of MLPSCs.

In an example, the culture medium is an above referenced composition or media. In an example, the culture medium is supplemented with a serum comprising one or more pro-inflammatory cytokines as described herein. In some preferred embodiments the culture medium to be used is supplemented with newborn serum. In some preferred embodiments the culture medium to be used is supplemented with both fetal serum and newborn serum in equal concentrations for a total serum concentration in the culture medium of about 10% (v/v). In some preferred embodiments MLPSCs are pre-licensed in cell culture medium containing 5% (v/v) newborn serum and 5% (v/v) fetal serum.

In some embodiments the methods disclosed herein include the additional step of determining or having determined the level of one or more pro-inflammatory cytokines in a serum to be included in the culture medium to be used for pre-licensing of MLPSCs. Methods for determining cytokine levels are well known in the art, e.g., ELISA.

In some embodiments the methods disclosed herein also include determining or having determined the ability of a culture medium (e.g. a newborn serum supplemented culture medium) to stimulate MLPSCs to promote angiogenesis in an in vitro assay, e.g., tube formation by human umbilical vein endothelial cells (HUVEC) and analysis of network length, network area and branch point formation. In some embodiments such an assay includes collecting MLPSC-conditioned media following its culture in a newborn serum-supplemented medium as disclosed herein and quantifying the effect of such conditioned media in the above-described angiogenesis assay or a similar assay.

In some embodiments the methods disclosed herein also include determining or having determined in the above-mentioned conditioned medium the level of one or more of Angiogenin, Angiopoietin (Ang1/ANGPT1), SDF-1α, and VEGF.

In some embodiments, where a first lot or batch of newborn serum was used in conditioned medium that promotes greater angiogenesis or release of angiogenic factors than that of conditioned medium in which second lot/batch of newborn serum was used, it is concluded that the use of the first lot of newborn serum for pre-licensing and culture expansion of MLPSCs will result in the generation of MLPSCs that have relatively greater therapeutic potency particularly for treatment of conditions where an angiogeneic or anti-inflammatory therapeutic mode of action is useful.

The methods and cell culture media of the present disclosure promote stem cell proliferation and pre-licensing while maintaining MLPSCs in an undifferentiated state. MLPSCs are considered to be undifferentiated when they have not committed to a specific differentiation lineage. As discussed above, MLPSCs display morphological characteristics that distinguish them from differentiated cells. Furthermore, undifferentiated MLPSCs express genes that may be used as markers to detect differentiation status. The polypeptide products may also be used as markers to detect differentiation status. Accordingly, one of skill in the art could readily determine whether the methods of the present disclosure maintain MLPSCs in an undifferentiated state using routine morphological, genetic and/or proteomic analysis. Methods of monitoring/confirming cell proliferation are also known in the art and, in certain examples, may be as rudimentary as periodic visual inspection of cell cultures to confirm increase in cell number. Other methods may involve the use of cell viability dyes and/or live cell imaging and counting using commercially available products.

MLPSCs disclosed herein can be culture expanded in various suitable cell culture mediums comprising newborn serum. The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Exemplary gaseous media include the gaseous phase that cells growing on a petri dish or other solid or semisolid support are exposed to.

In an example, the methods of the disclosure encompass culture expansion in cell culture media which comprise one or more pro-inflammatory cytokines. In an example, the cell culture media comprises IFN-gamma and/or TNF-alpha. In an example, the cell culture media comprises IFN-gamma. For example, the level of IFN-gamma can be less than 1 ng/ml. In an example the level of IFN-gamma is less than 500 pg/ml or less than 100 pg/ml. In an example, the cell culture media comprises TNF-alpha. For example, the level of TNF-alpha can be less than 1 ng/ml. In an example, the level of TNF-alpha is less than 750 pg/ml or less than 400 pg/ml. In an example, the cell culture media comprises IFN-gamma and TNF-alpha and the level of both is less than 1 ng/ml.

In an example, the cell culture media comprises one or more pro-inflammatory cytokines which are capable of binding a receptor on the surface of MLPSCs.

In an example, the cell culture media comprises one or more pro-inflammatory cytokines selected from the group consisting of IL-6; IL-8; IL-17A; MCP-1; MIP-1-alpha; MIP-1-beta; IP-10. For example, the cell culture media can comprise IL-8.

In an example, the cell culture media comprises IFN-gamma and/or TNF-alpha, and, one or more pro-inflammatory cytokines selected from the group consisting of IL-6; IL-8; IL-17A; MCP-1; MIP-1-alpha; MIP-1-beta; IP-10. In an example, the level of IFN-gamma and/or TNF-alpha is less than 1 ng/ml.

In an example, the cell culture media is characterised by one or more or all of the following:
1. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

In another example, the media comprises serum characterised by one or more or all of the following:
i. a level of IFN-gamma greater than 10 pg/ml;
ii. a level of TNF-alpha greater than 20 pg/ml;
iii. a level of IL-6 greater than 30 pg/ml;
iv. a level of IL-8 greater than 5,000 pg/ml;
v. a level of IL-17A greater than 2 pg/ml;
vi. a level of MCP-1 greater than 30 pg/ml;
vii. a level of MIP-1-alpha greater than 50 pg/ml;
viii. a level of MIP-1-beta greater than 30 pg/ml;
ix. a level of IP-10 greater than 5,000 pg/ml.

In an example, the media comprises IL-10. In another example, the media comprises IL-36RA. In another example, the media comprises IL-10 and IL-36RA. In an example, the level of IL-10 is greater than 0.3 pg/ml. For example, the level of IL-10 may be greater than 30 pg/ml. In an example, the level of IL-10 is greater than 400 pg/ml. In an example, the level of IL-36RA is greater than 50 pg/ml.

In another example, methods of the disclosure encompass culture expansion in cell culture media which comprise newborn serum. Various examples of suitable serum (and levels of the same) are disclosed herein.

In an example, methods of the disclosure comprise selecting a cryopreserved intermediate population of MLPSCs for culture expansion in media disclosed herein. In an example, a cryopreserved intermedia that has been culture expanded in 10% fetal serum is selected for culture expansion according to the methods disclosed herein. In an example, a cryopreserved intermediate that has been culture expanded in newborn serum and/or pro-inflammatory cytokines disclosed herein is selected for culture expansion.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

### Serum

“Newborn serum” refers to serum that has been obtained postpartum. For example, the culture media can be supplemented with mammalian newborn serum (e.g. bovine). In an example, the culture media can be supplemented with animal newborn serum. In another example, the culture media can be supplemented with human newborn serum.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v newborn serum. In an example, the cell culture media is supplemented with between about 1% v/v and about 15% v/v newborn serum. In an example, the cell culture media is supplemented with between about 1% v/v and about 10% v/v newborn serum. In an example, the cell culture media is supplemented with between about 5% v/v and about 10% v/v newborn serum. In an example, the cell culture media is supplemented with about 5% v/v newborn serum.

In an example, the newborn serum comprises at least one inflammatory cytokine. As used herein, the term “inflammatory cytokine” refers to a signalling molecule that promotes inflammation. In example, the one or more cytokine is selected from the group comprising IL-1β, IL-6, TNF-α, IFN-γ and/or IL-1ra.

In an example, the newborn serum comprises IFN-gamma. In another example, the newborn serum comprises TNF-alpha. In another example, the newborn serum comprises IFN-gamma and TNF-alpha. In another example, the newborn serum comprises one or more pro-inflammatory cytokines selected from the group consisting of IL-6; IL-8; IL-17A; MCP-1; MIP-1-alpha; MIP-1-beta; IP-10. For example, the newborn serum can comprise IL-8. In an example, the newborn serum comprises IFN-gamma and/or TNF-alpha and, one or more pro-inflammatory cytokines selected from the group consisting of IL-6; IL-8; IL-17A; MCP-1; MIP-1-alpha; MIP-1-beta; IP-10. In another example, the newborn serum comprises IFN-gamma and TNF-alpha and, one or more pro-inflammatory cytokines selected from the group consisting of IL-6; IL-8; IL-17A; MCP-1; MIP-1-alpha; MIP-1-beta; IP-10. In an example, the level of IFN-gamma is less than 1 ng/ml. In an example, the level of TNF-alpha is less than 1 ng/ml. In an example, the level of both IFN-gamma and TNF-alpha are less than 1 ng/ml. For example, the level of IFN-gamma may be less than 500 pg/ml or less than 100 pg/ml. In an example, the level of TNF-alpha is less than 750 pg/ml or less than 400 pg/ml.

Methods to detect the presence of cytokines in serum are known in the art and include, for example, enzyme-linked immunosorbent assay (ELISA). In another example, the presence of cytokines in serum are detected by measuring cytokine mRNA, for example by polymerase-chain reaction (PCR) techniques such as reverse-transcription PCR.

In an example, the newborn serum can be newborn calf serum (NBCS). In an example, the NBCS is obtained from newborn calves who have been fed colostrum. In an example, NBCS comprises elevated levels of at least one inflammatory cytokine relative to NBCS obtained from a calf that has not been fed colostrum. In an example, NBCS comprises elevated levels of at least one inflammatory cytokine relative to fetal serum such as FCS.

In an example, the NBCS is obtained within 4 weeks after birth of the calf. In an example, the NBCS is obtained within 21 days after birth of the calf. For example, the NBCS is obtained ≤21 days after birth of the calf. In an example, the NBCS is obtained between the day of birth and 21 days after birth of the calf. In an example, the NBCS is obtained between the day of birth and 14 days after birth of the calf. In an example, the NBCS is obtained between the day of birth and 10 days after birth of the calf. In an example, the NBCS is obtained between the day of birth and 7 days after birth of the calf. In an example, the NBCS is obtained between 6 hours after birth and 72 hours after birth. In an example, the NBCS is obtained between 6 hours after birth and 48 hours after birth. In an example, the NBCS is obtained between 6 hours after birth and 24 hours after birth. In an example, the NBCS is obtained between 12 hours after birth and 24 hours after birth.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v NBCS. In an example, the cell culture media is supplemented with between about 1% v/v and about 15% v/v NBCS. In an example, the cell culture media is supplemented with between about 5% v/v and about 10% v/v NBCS. In an example, the cell culture media is supplemented with at least about 5% v/v NBCS.

In an example, the culture medium is also supplemented with fetal serum. In an example, the fetal serum is fetal calf serum (FCS). It is envisaged that the term fetal calf serum (FCS) and fetal bovine serum (FBS) can in the context of the present disclosure be used interchangeably. In an example, cell culture medium is supplemented with less than 10% v/v FCS. In an example, cell culture medium is supplemented with about 5% v/v FCS.

In an example, the cell culture medium is fetal serum free.

In an example, the cell culture medium is FCS free.

In an example, the culture media is supplemented with a mixture of FCS and NBCS. In an example the cell culture medium is supplemented with about 5% v/v FCS and about 5% v/v NBCS (i.e. a 1:1 ratio of FCS to NBCS). In an example, the culture media can be supplemented with a mixture of FCS and NBCS so that the FCS:NBCS ratio is at least about 0.4:1, at least about 0.5:1, at least about 0.6:1, at least about 0.7:1, at least about 0.8:1, at least about 0.9:1, at least about 1:1, at least about 1.5:1, at least about 2:1. In an example, the FCS:NBCS ratio is between about 0.5:1 and about 2:1. In an example, the FCS:NBCS ratio is between about 0.8:1 and about 1.5:1. In an example, the FCS:NBCS ratio is between about 0.8:1 and about 1.2:1. In an example, the FCS:NBCS ratio is about 1:1.

In an example, the mixture of FCS and NBCS can comprise at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v of the cell culture media. In an example, the mixture of FCS and NBCS can comprise between about 1% v/v and about 15% v/v of the cell culture media. In an example, the mixture of FCS and NBCS can comprise between about 2% v/v and about 12% v/v of the cell culture media. In an example, the mixture of FCS and NBCS can comprise between about 5% v/v and about 12% v/v of the cell culture media. In an example, the mixture of FCS and NBCS can comprise between about 8% v/v and about 12% v/v of the cell culture media. In an example, the mixture of FCS and NBCS can comprise about 10% v/v of the cell culture media However, in this example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v, but less than 10% v/v FCS. In an example, the cell culture media is supplemented with between about 1% v/v and about 9% v/v FCS. In an example, the cell culture media is supplemented with between about 3% v/v and about 8% v/v FCS. In an example, the cell culture media is supplemented with between about 3% v/v and about 6% v/v FCS. In an example, the cell culture media is supplemented with about 5% v/v FCS.

### Ascorbic Acid

In an example, the cell culture media is supplemented with a short acting ascorbic acid derivative. The term “short acting” encompasses ascorbic acid derivatives that are oxidised by approximately 80-90 % following 24 hours of cell culture under culture conditions of neutral pH and 37° C. In one example, the short acting L-ascorbic acid derivative is a L-ascorbic acid salt, for example L-ascorbic acid sodium salt. In an example, the cell culture media may contain at least about 0.005 g/L of a short acting ascorbic acid derivative. In another example, the cell culture media may contain at least about 0.01 g/L of a short acting ascorbic acid derivative. For example, the cell culture media may contain at least about 0.02 g/L of a short acting ascorbic acid derivative. In another example, the cell culture media may contain at least about 0.03 g/L of a short acting ascorbic acid derivative. For example, the cell culture media may contain at least about 0.04 g/L of a short acting ascorbic acid derivative. In another example, the cell culture media may contain at least about 0.05 g/L of a short acting ascorbic acid derivative. In another example, the cell culture media may contain at least about 0.06 g/L of a short acting ascorbic acid derivative.

In another example, the cell culture media contains a short acting ascorbic acid derivative but does not contain a substantial amount of a long acting ascorbic acid derivative. For example, the cell culture media may contain a short acting ascorbic acid derivative but not more than 0.04 g/L of a long acting ascorbic acid derivative. In another example, the cell culture media may contain a short acting ascorbic acid derivative but not more than 0.03 g/L of a long acting ascorbic acid derivative. In another example, the cell culture media may contain a short acting ascorbic acid derivative but not more than 0.02 g/L of a long acting ascorbic acid derivative. In another example, the cell culture media may contain a short acting ascorbic acid derivative but not more than 0.01 g/L of a long acting ascorbic acid derivative. In another example, the cell culture media may contain a short acting ascorbic acid derivative but not more than 0.005 g/L of a long acting ascorbic acid derivative. In another example, the cell culture media may contain a short acting ascorbic acid derivative but not a long acting ascorbic acid derivative. In another example, the cell culture media contains L-ascorbate sodium salt but does not contain a substantial amount of L-ascorbic acid-2-phosphate.

### Other Additives

In an example, the cell culture medium contains human derived additives. For example, human serum and human platelet cell lysate can be added to the cell culture media. In other examples, additional factors can be added to the cell culture medium. For example, the cell culture media can be supplemented with one or more stimulatory factors selected from the group consisting of, platelet derived growth factor (PDGF), fibroblast growth factor 2 (FGF2), epidermal growth factor (EGF), epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof.

In the above examples, basal medium such as Alpha MEM or StemSpan™ can be supplemented with the referenced quantity of serum and, in certain examples, other additives. Further examples of suitable culture mediums for culturing stem cells can be found, for example, in WO2016139340.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from U.S. 63/386,876 filed 9 Dec. 2022 and U.S. 63/507,009 filed 8 Jun. 2023, the disclosures of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### EXAMPLES

### Example 1: Serum Analysis

Mesenchymal precursor lineage or stem cell populations were culture expanded in either 5% FCS/5% NBCS (serum A) or 10% fetal bovine serum (serum B). These MLPSCs were used in examples 4-6.

Cytokine levels in 5% FCS/5% NBCS (serum A) and 10% fetal bovine serum (serum B) were assessed. To provide an external control, cytokine levels were also assessed in FBS from a different supplier (serum C). In each instance, cytokine levels were assessed in neat serum.

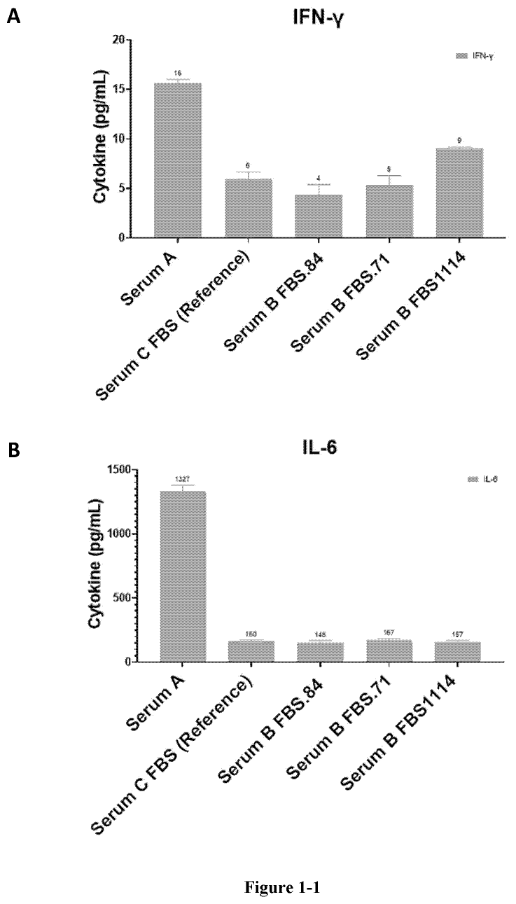

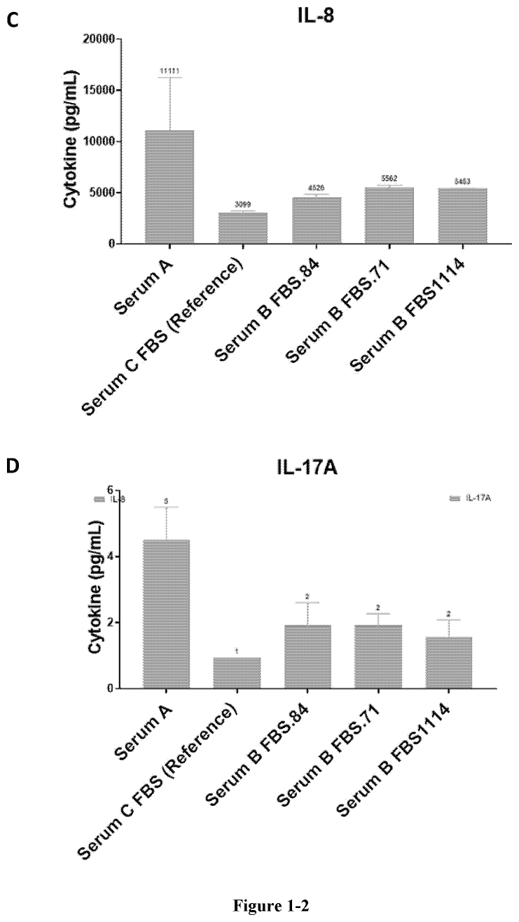

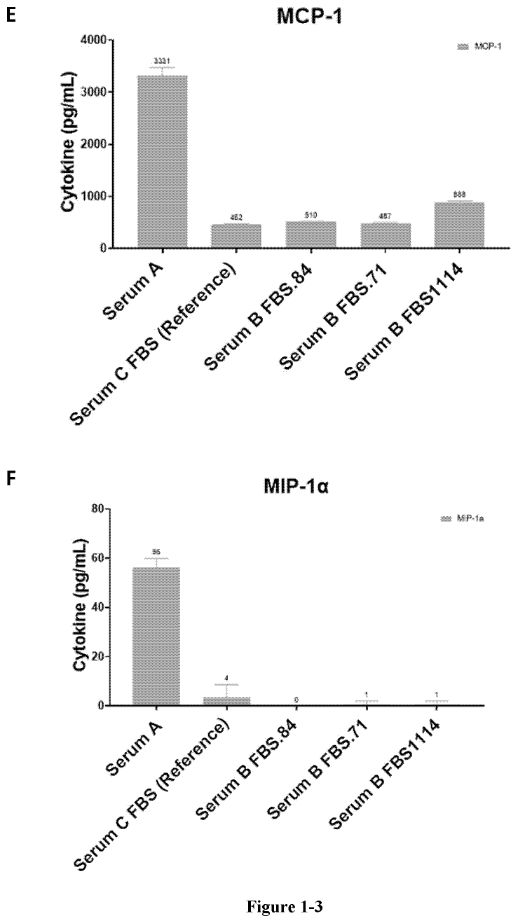

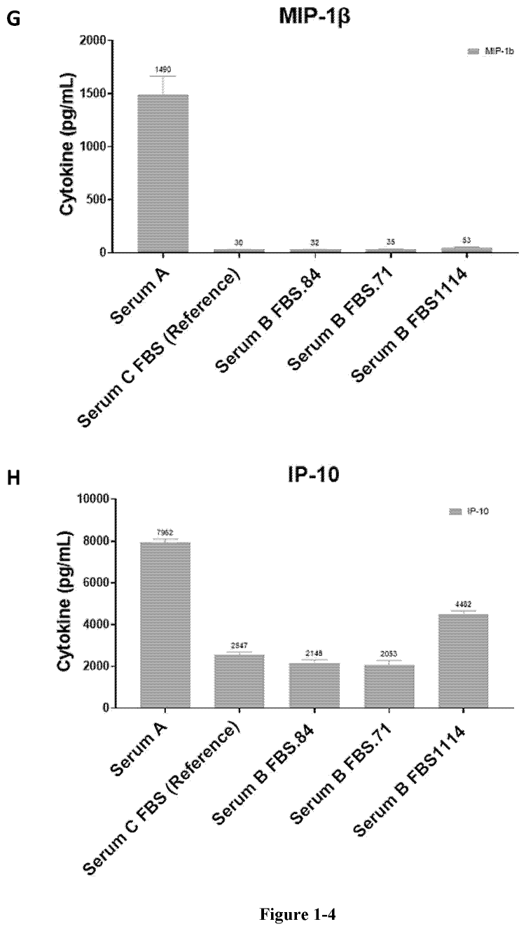

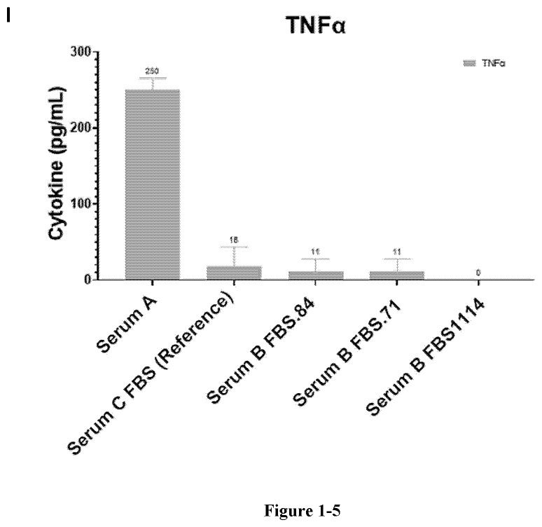

Surprisingly, pro-inflammatory cytokine levels were higher in serum preparations containing newborn calf serum (FIG. 1). Notably, there was an increase in pro-inflammatory cytokines known to bind receptors expressed on the surface of MLPSCs, such as interferon gamma (IFNγ), tumor necrosis factor alpha (TNFα) and, interleukins. For example, the following was observed in serum preparations containing newborn calf serum relative to fetal bovine serums:
At least a 2× increase in IFNγ;
At least an 13× increase in TNFα;
At least an 8× increase in IL-6;
At least an 2× increase in IL-8;
At least an 2× increase in IL-17A.

### Example 2: MLPSC Compositions Derived Using Culture Media Comprising Fetal Serum

The Alpha modification of Eagle's minimum essential media (MEM) with Earle's balanced salts, commonly referred to as Eagle's Alpha MEM, contains non-essential amino acids, sodium pyruvate, and additional vitamins. These modifications were first described for use in growing hybrid mouse and hamster cells (Stanners et al. 1971).

Eagle's Alpha MEM media suitable for culturing primary stem cells can be obtained from a variety of sources, including Life Technologies and Sigma.

A detailed method of establishing primary stem cell cultures, including the required growth factors used in the Exemplified processes is described in Gronthos and Simmons 1995.

Eagle's Alpha MEM media supplemented with 10% fetal calf serum (serum B), L-ascorbate-2-phosphate (100 μM), dexamethasone (10⁻⁷ M) and/or inorganic phosphate (3 mM) was used for culturing MLPSCs.

### Example 3: MLPSC Compositions Derived Using Culture Media Comprising Newborn Serum

For the MLPSC culture media comprising newborn serum, the serum component of the Eagle's Alpha MEM culture media described in Example 2 was modified by supplementing with 5% (v/v) newborn serum (Differences in the fetal serum media and newborn serum media are shown in Table 1). The newborn serum used was newborn calf serum (NBCS; serum A). NBCS was 100% bovine serum obtained from animals meeting the standard fetal bovine serum specifications but under the age of 20 days after birth.

NBCS was obtained from a commercial supplier, where it is marketed as an FCS substitute that is highly similar to FCS, to be used interchangeably, and expected to perform the same on cell lines.

TABLE 1
Summary of the differences between fetal serum
culture media and licensing culture media

| | |
|---|---|
| Fetal serum | |
| culture media | Licensing culture media |

Media (Change applicable to Thaw Feed, Passage)

| | |
|---|---|
| Alpha MEM | 50 mg/L short acting ascorbic acid derivative |
| 10% v/v FCS | (Sodium L-ascorbate) replaces long acting ascorbic |
| | acid derivative (L-ascorbic acid-2-phosphate) |
| | 5% v/v FCS and 5% v/v newborn serum |

Cryopreservation Formulation
(50% Alpha-MEM/42.5% ProFreeze/7.5% DMSO)

| | |
|---|---|
| Alpha MEM | 50 mg/L short acting ascorbic acid derivative |
| 10% v/v FCS | (Sodium L-ascorbate) replaces long acting ascorbic |
| | acid derivative (L-ascorbic acid-2-phosphate) |
| | 5% v/v FCS |
| | 5% v/v newborn calf serum |

### Example 4: Culture Expansion of MLPSCs in Media Supplemented with Newborn Serum Enhances Angiogenesis

With a view to characterising the novel MLPSC populations derived by culture expansion in media supplemented with newborn serum and/or pro-inflammatory cytokines (and to potentially identify a mechanism for the observed increase in therapeutic efficacy described in Example 5), we assessed the angiogenic potential of MPCs cultured under different conditions.

Cell culture: MPCs were cultured with either 5% NBCS/5% FCS (serum A) or 10% FCS (serum B) to generate MPC-conditioned media. To control for donor variation, MPCs were obtained from the same donor and then cultured under different conditions. In some experiments, MPCs belonging to same donor but cultured during a different manufacturing expansion are indicated by separate “lot” numbers.

Conditioned media was obtained by separating the cells from the culture media supernatant. Briefly, cryopreserved MPCs were thawed and seeded at 50,000/cm² in alpha MEM and either 10% FBS or 5% NBCS/5% FCS. Conditioned media (CM) was collected after incubating the cells for 72 h at 37° C. 5% CO₂. VEGF, SDF-1 and angiogenin levels in CM were measured using Luminex (R&D Systems). The CM was concentrated using a 3k protein concentration filtration column (Amicon® Ultra-15) and reconstituted back to 1× or 0.25× in Assay medium.

Angiogenesis potency assay: In-vitro angiogenesis was measured using a kinetic, quantitative 96-well co-culture angiogenesis model. Lentivirus-transduced human umbilical vein endothelial cells (HUVEC) expressing CytoLight Green (a GFP variant) cultured with normal human dermal fibroblasts (NHDF) were seeded into 96-well plates and simultaneously incubated and imaged using the IncuCyte® Live-Cell Analysis System. This system enables the fluorescent identification of HUVEC (CytoLight Green⁺) cells and allows visualization of tube formation over time by time-lapse image acquisition. The acquired images are analysed using an integrated angiogenesis algorithm to measure network length, network area and branch point formation to provide a quantitation of the stage and extent of angiogenesis throughout the assay.

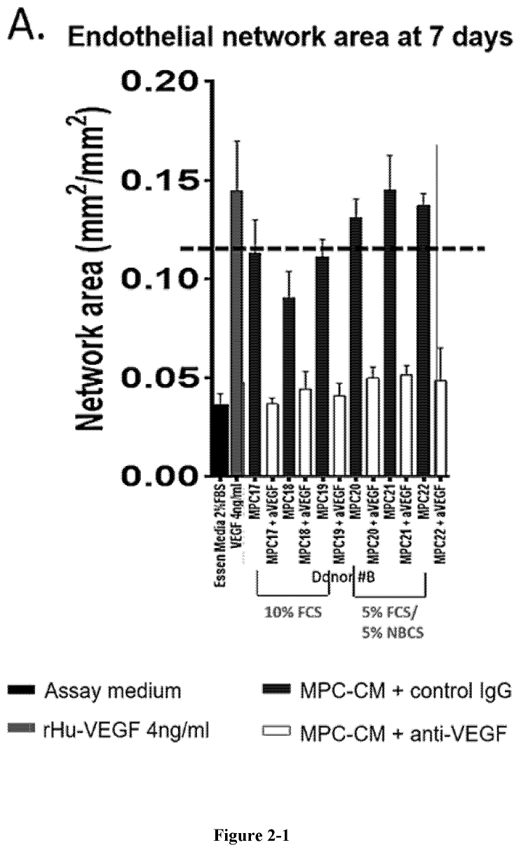

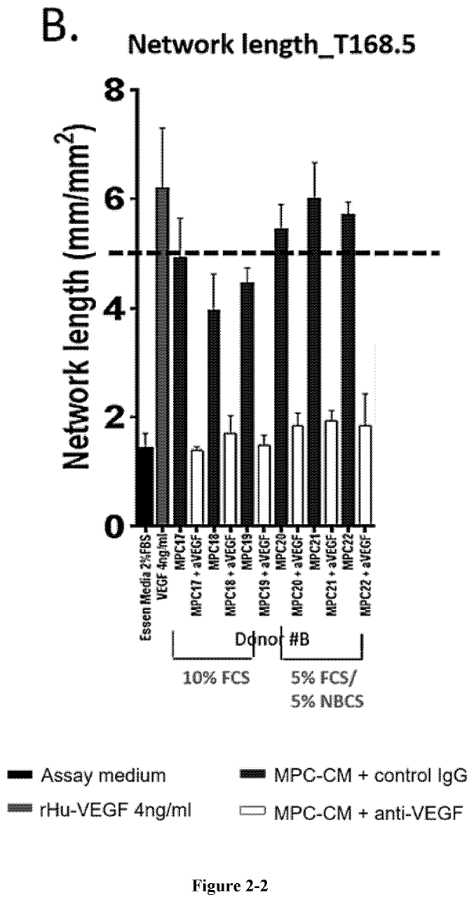

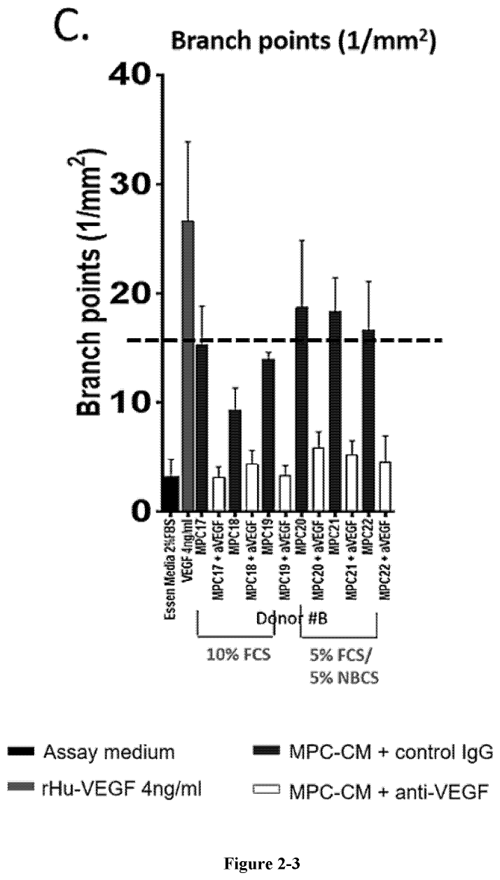

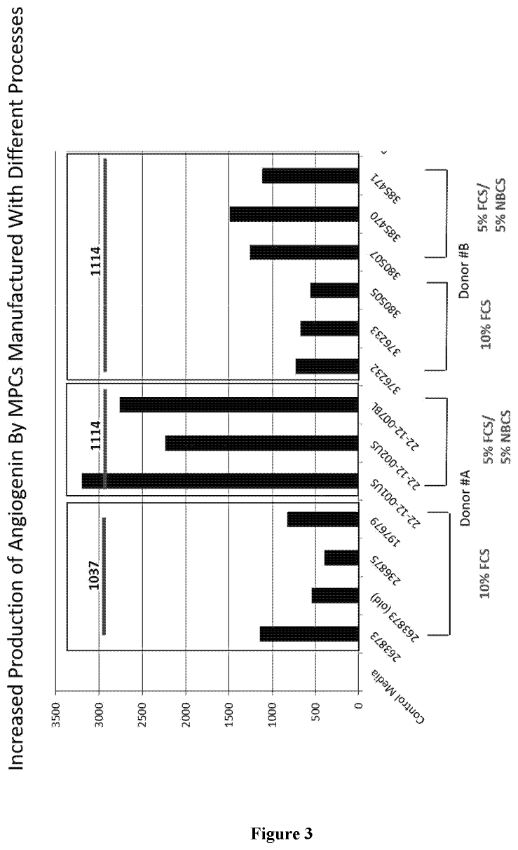

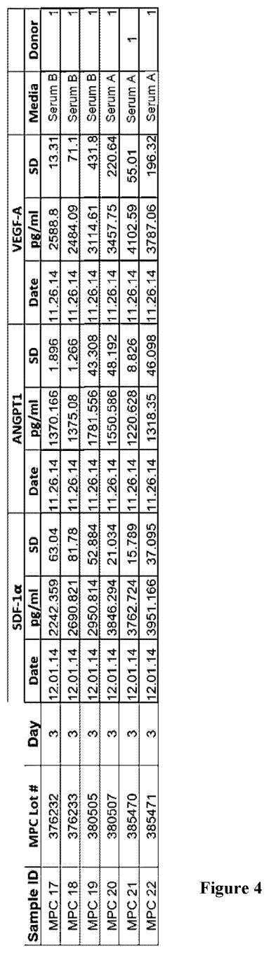

Results: Conditioned media from MPCs cultured in media supplemented with newborn calf serum was found to increase angiogenesis. As shown in FIG. 2, conditioned media from MPCs cultured in 5% NBCS/5% FCS increased network area (FIG. 2A), network length (FIG. 2B) and branch points (FIG. 2C) in the co-culture angiogenesis model. Furthermore, conditioned media from MPCs cultured in 5% NBCS/5% FCS comprises higher levels of VEGF compared to conditioned media from cells cultured in 10% FCS (FIG. 2A). Angiogenin levels were also increased in conditioned media from MPCs cultured in 5% NBCS/5% FCS when compared to 10% FCS (FIG. 3). FIG. 4 shows further analysis of the levels of angiogenic factors SDF-1α, VEGF and Ang1 (ANGPT1) present in MPCs cultured in either 10% FCS (“serum B Media”) or 5% FCS/5% N BCS (“serum A Media”). These data show that both VEGF and SDF-1α are elevated in newborn serum media-cultured MPCs.

In view of the data provided in Example 1, these data indicate that culture expansion of MLPSCs in media supplemented with newborn serum and/or proinflammatory cytokines provides novel cell populations with enhanced angiogenic potential. This enhanced potential may be, if required, characterised in various ways (for example, to define the novel cell populations identified by the present inventors) including, for example:
Capacity for conditioned media derived from the MLPSCs to increase network area, network length and/or branch points when contacted with HUVECs;
Angiogenin, VEGF and/or SDF-1 levels in conditioned media.

### Example 5: MLPSCs Cultured in Media Supplemented with Newborn Serum Improve Therapeutic Outcomes in the Context of Persistent Inflammation

NYHA Class II/III high-risk heart failure with reduced ejection fraction (HFrEF) is a clinical model of persistent inflammation. HFrEF patients are characterized by cardiac and systemic inflammation, as determined by the presence of elevated inflammatory biomarkers. MPCs cultured under different serum conditions were administered to HFrEF patients in the clinical study described below.

In HFrEF patients, cardiac macrophages produce high levels of pro-inflammatory cytokines (IL-6, IL-1, TNF-alpha) which cause endothelial dysfunction and cardiomyocyte apoptosis. Plasma C-Reactive Protein (CRP) levels, as determined by a high sensitivity CRP (hsCRP) assay, reflect hepatic production of acute phase reactants in response to the high levels of pro-inflammatory cytokines (IL-6, IL-1 and TNF-alpha) produced by cardiac macrophages. Accordingly, plasma hsCRP levels (<2 mg/L vs >2 mg/L) are representative systemic measurements reflective of low or high intra-cardiac inflammation. In the following study, HFrEF patients were categorized as having persistent inflammation if their plasma hsCRP levels were >2 mg/L.

Study details: Eligible NYHA Class II/III patients were enrolled in the Double-blind, Randomized, Sham-procedure-controlled, Parallel-Group Efficacy and Safety Study of Allogeneic Mesenchymal Precursor Cells (Rexlemestrocel-L) in Chronic Heart Failure Due to LV Systolic Dysfunction (Ischemic or Nonischemic) (DREAM HF-1) trial. HFrEF patients were administered: (1) MPCs cultured in 10% fetal serum (n=37), (2) MPCs cultured in the presence of newborn calf serum (5% FCS/5% NBCS; n=153) or, (3) a sham control (i.e., no MPCs; n=241). As evidenced by the serum analysis described in Example 1, cells cultured in media supplemented with newborn serum were effectively cultured in media comprising increased levels of pro-inflammatory cytokines. Cells were administered in a single transendocardial injection. LV systolic function in HFrEF was measured by echocardiogram (ECHO) parameters including left ventricular ejection fraction (LVEF; %), left ventricular end-systolic volume (LVESV; mL), and left ventricular end-diastolic volume (LVEDV; mL) at baseline and 12 months post treatment. Plasma CRP levels were measured to determine baseline levels of inflammation.

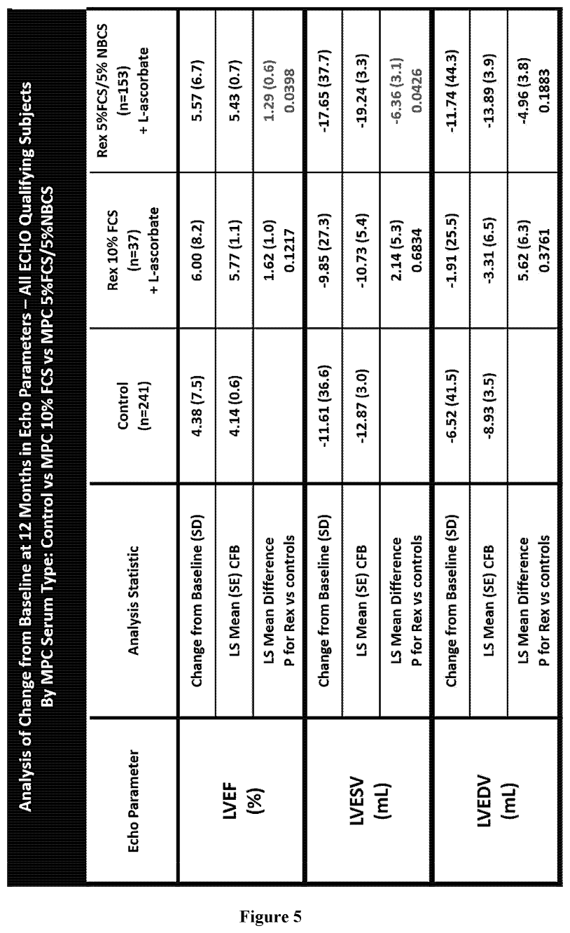

Results: MPCs cultured in the presence of newborn calf serum (5% FCS/5% NBCS) were found to improve left ventricular (LV) systolic function in HFrEF patients at 12 months. In particular, newborn serum-cultured MPCs significantly increased LVEF and decreased LVESV compared to sham controls (p=0.0398 and 0.0426, respectively) (FIG. 5).

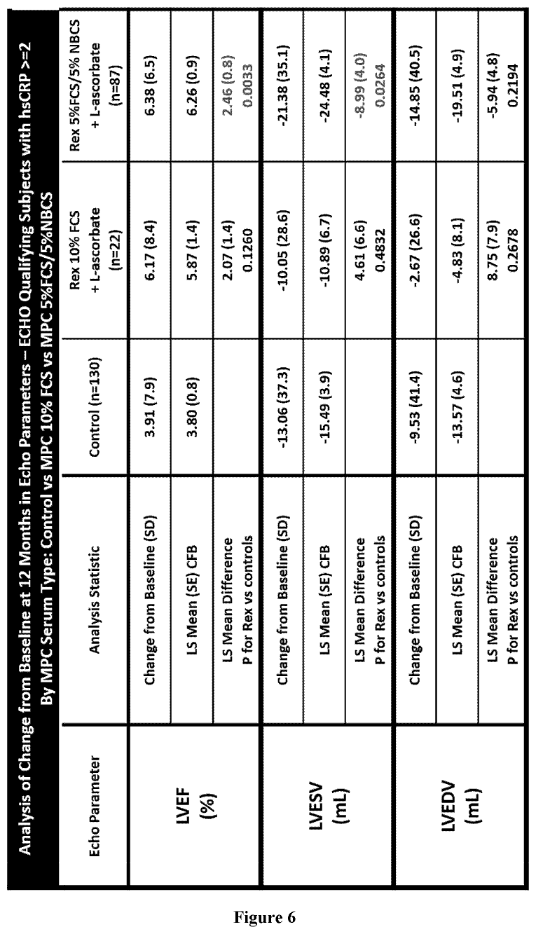

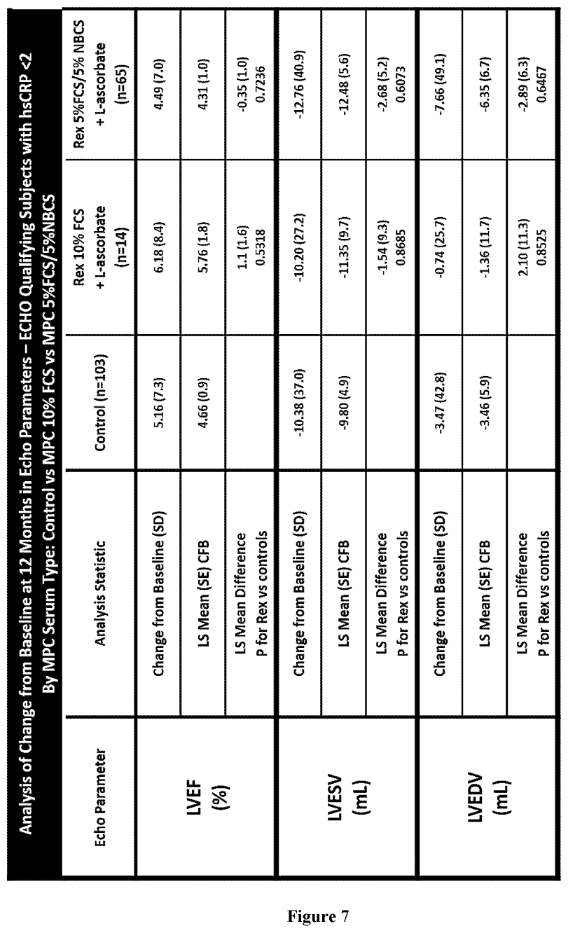

HFrEF patients were then characterised according to plasma hsCRP levels of either <2 mg/L (normal baseline systemic inflammation) or >2 mg/L (elevated baseline systemic inflammation). Importantly, when HFrEF patients were differentiated according to baseline systemic inflammation status (CRP >2), the effect of treatment with MPCs cultured in the presence of newborn calf serum (5% FCS/5% NBCS) LV systolic functional recovery induced was more pronounced. In contrast, MPCs cultured in 10% FBS did not induce a significant effect (FIG. 6). Newborn serum-cultured MPCs significantly increased LVEF % by an average (LS mean) of 2.46 and decreased LVESV by an average of 8.99 mL compared to sham controls (p=0.0033 and 0.0264, respectively). In contrast, MPCs cultured in 10% fetal serum or 5%/FCS/5% NBCS showed improvements to LV systolic function in HFrEF patients without elevated baseline inflammation (HFrEF patients with CRP <2) (FIG. 7).

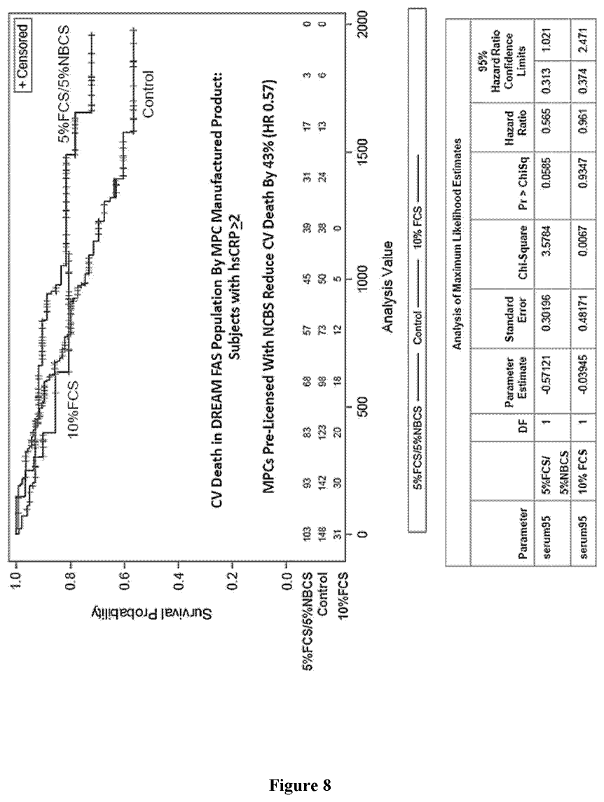

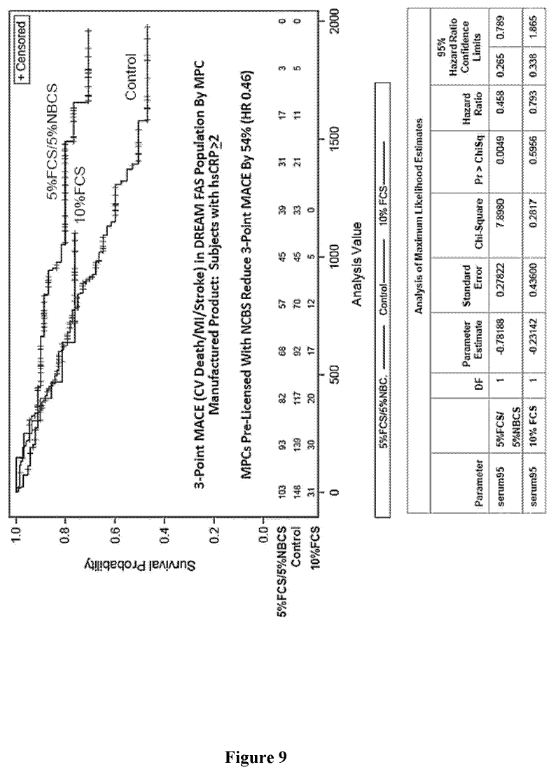

MPCs cultured in media supplemented with newborn serum were also found to reduce other cardiac outcomes in HFrEF patients with CRP>2, including reducing the risk of cardiovascular death by 43% (FIG. 8) and incidence of 3-point MACE (CV Death/MI/Stroke) by 54% (FIG. 9). These data show that MPCs cultured in media supplemented with newborn serum provide improved therapeutic efficacy in the context of persistent inflammation.

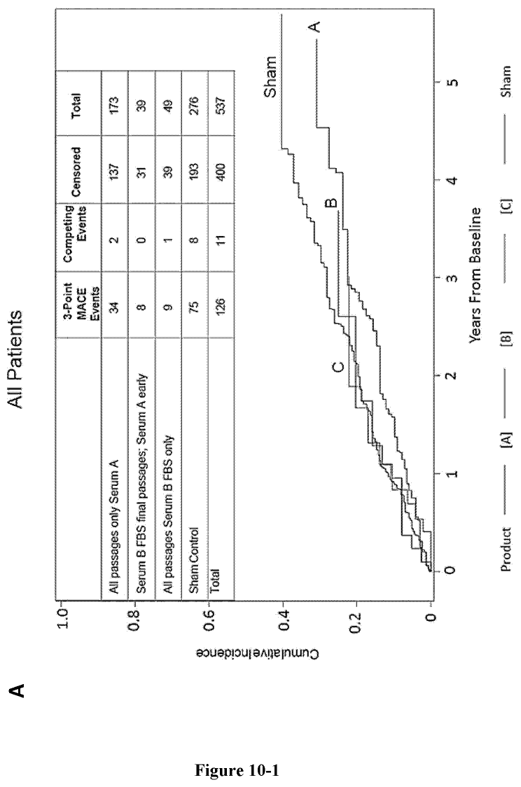

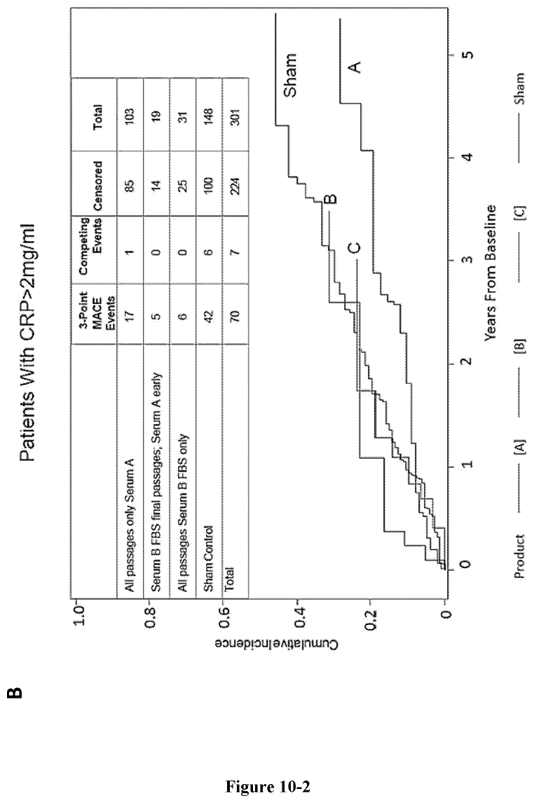

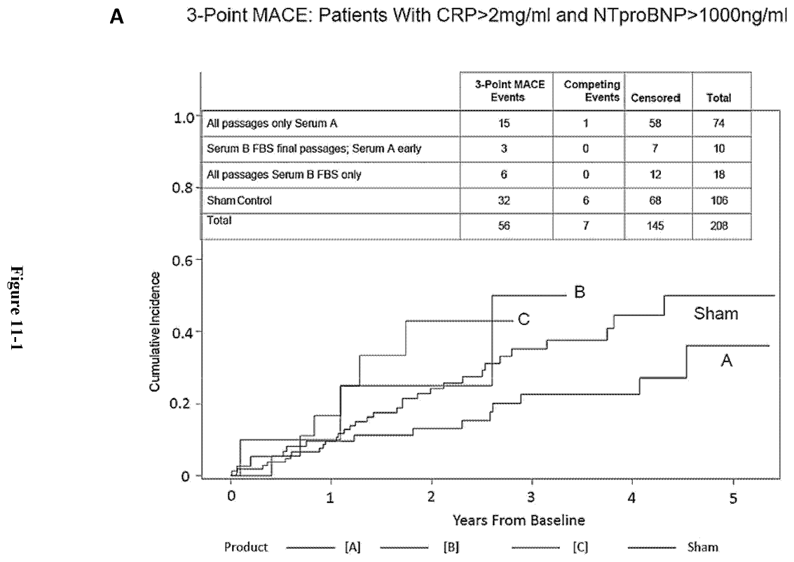

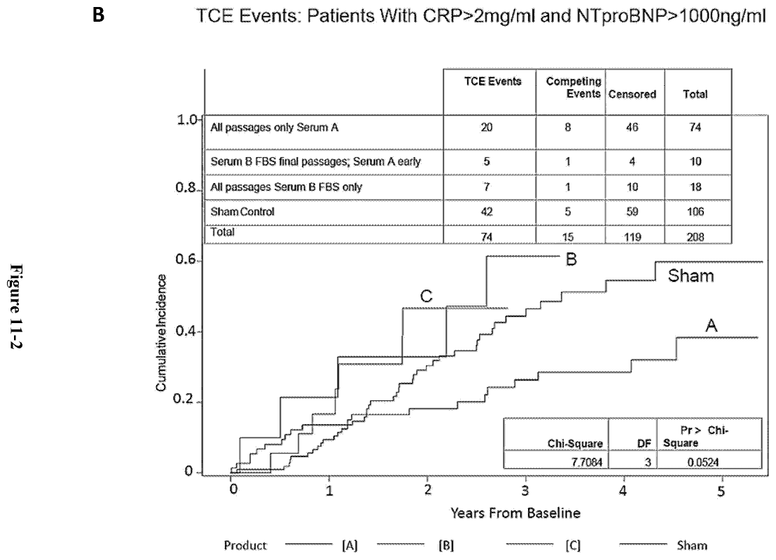

Further analysis of clinical response surprisingly revealed the importance of culture expanding MLPSCs in media supplemented with newborn serum and/or pro-inflammatory cytokines during final passage(s) before administration. MLPSCs cultured in media supplemented with newborn serum and/or pro-inflammatory cytokines during final passages reduced 3-Point MACE (MI, Stroke or CV Death) in patients, irrespective of whether the MLPSCs were culture expanded in FBS during earlier passages. Surprisingly, this reduction in 3-point MACE was observed in all patients, regardless of inflammation status (FIG. 10). Patient sub group analysis revealed that the reduction in 3-point MACE was observed in patients with persistent inflammation (CRP>2 mg/ml; FIG. 10) and, patients in the study at highest risk of Terminal Cardiac Events (TCE; defined as CV Death, Heart Transplant or Left Ventricular Assists Device Implantation). The patients at highest risk of TCE are defined by both CRP>2 mg/ml and NT-proBNP >1000 ng/ml; FIG. 11)).

In contrast, increased adverse events (3-Point MACE) were observed in all patients administered MLPSCs that were culture expanded in media that was not supplemented with newborn serum and/or pro-inflammatory cytokines during final passages before administration. The increase in 3-point MACE was observed regardless of whether the MLPSCs were cultured in media supplemented with newborn serum and/or pro-inflammatory cytokines during earlier passages.

Summary: MPCs expanded in media supplemented with newborn calf serum (NBCS) and/or pro-inflammatory cytokines:
improved left ventricular systolic dysfunction in HFrEF patients with inflammation, as measured by LS mean change in LVEF and LVESV at 12 months;
reduced cardiovascular death by 43% in high-risk NYHA Class II/III patients with HFrEF and inflammation; and,
reduced long-term 3 Point MACE by 54% in high-risk NYHA Class II/III patients with HFrEF and inflammation.

Taken together with the results in Examples 1 and 4, these human trial data indicate that supplementing cell culture media with newborn serum and/or pro-inflammatory cytokines provides a cell population with different functional characteristics, at least in terms of capacity to direct therapeutic efficacy in an inflammatory environment.

These human trial data also evidence the importance of supplementing media with newborn serum and/or pro-inflammatory cytokines during final passage(s), for example, during culture expansion of intermediate cell populations to drug product. Accordingly, the present inventors findings underpin at least two approaches to providing MLPSC populations with improved therapeutic efficacy for administration to patients:
Expansion of MLPSCs to intermediate population in newborn serum and/or pro-inflammatory cytokines to provide cryopreserved intermediate population; expansion of cryopreserved intermediate population in newborn serum and/or pro-inflammatory cytokines to provide drug product.
Expansion of MLPSCs to intermediate population in fetal serum to provide cryopreserved intermediate population; expansion of cryopreserved intermediate population in newborn serum and/or pro-inflammatory cytokines to provide drug product.

Without wishing to be bound by any particular theory, the above referenced data suggest that culturing in media supplemented with newborn serum and/or pro-inflammatory cytokines pre-licenses MPCs to respond more effectively to inflammatory environments. Furthermore, taken together with the results described in Example 4, these data point to a potential mechanism by which MPCs cultured in media supplemented with newborn serum and/or pro-inflammatory cytokines impart improved therapeutic efficacy, namely, the enhanced angiogenesis and increased production of pro-angiogenic growth factors, VEGF, SDF-1α and angiogenin. Accordingly, these data provide basis for a method of selecting cells with a sufficient potency for the treatment of inflammatory disorders. In particular, the data shows that a threshold level of >3.45 ng/ml VEGF, >3000 ng/ml SDF-1α, or >1114 pg/mL angiogenin, with concentrations in advance of these amounts indicating therapeutic potency and increased biological activity of MPCs. For example, cells can be cultured according to the methods disclosed herein, conditioned media could be harvested and measured in the angionesis assay and/or for levels of VEGF and angiogenin. Cells which produce VEGF/angiogenin above the threshold are considered therapeutically potent/biologically active. Similarly, cells which produce conditioned media that enhances angiogenesis as determined by a network area of >0.12 mm²/mm², network length of >5 mm²/mm² and/or branch points of >15 mm²/mm² are also considered to be therapeutically potent/biologically active for treating inflammatory diseases.

The above referenced angiogenic markers may be relevant criteria (in addition to the clinical response criteria shown in heart failure) for characterising the novel MLPSCs produced via culture in cell culture media comprising newborn serum and/or pro-inflammatory cytokines.

### Example 6: MLPSCs Cultured in Media Supplemented with Newborn Fetal Serum are Effective in Treating GvHD

GvHD patients were administered (intravenous) MPCs culture expanded with NBCS containing pro-inflammatory cytokines (Examples 2 and 4) once per week at a dose of 2×10⁶ MPCs per kg. Patient response is summarised in Table 2. 80% of GvHD patients administered MPCs cultured in NBCS responded to treatment. 1 patient achieved complete response, 7 patients achieved partial response and 2 patients died.

TABLE 2
GvHD response to MPCs culture expanded with NBCS

| | Non-fetal |
|---|---|
| | serum |
| Complete Response | 1 |
| (CR) | |
| Partial Response (PR) | 7 |
| Died | 2 |
| No Response (NR) | 0 |
| Total | 10 |

Taken together with the results of at least Example 6, the findings of the present inventors underpin broad application of pre-licensed MLPSCs (e.g. MPCs cultured in the presence of non-fetal serum, in particular newborn serum and/or presence of pro-inflammatory cytokines) for treatment of any disease or disorder characterised by elevated inflammation, in particular diseases characterized by persistent inflammation such as heart failure or T-cell mediated disorders such as GvHD.

### Example 7: Media Analysis and Summary of Findings

Based on the data described in Example 1, cytokine levels were increased in culture medium used to expand MLPSC populations characterised by increase(s) in one or more angiogenic markers (example 4) and increased therapeutic efficacy in both heart failure (example 5) and GvHD (example 6) patients. The correlation between increased pro-inflammatory cytokine levels in culture media and therapeutic efficacy in separate disease indications associated with inflammation suggests a pre-licensing effect on MLPSCs.

Surprisingly, MLPSCs described herein appear to have been pre-licensed by culture with pro-inflammatory cytokines, despite these cytokines being present at very low levels (e.g. pg/ml levels). This is surprising because it was not previously envisaged that pro-inflammatory cytokines, in particular TNF-alpha and IFN-gamma, could have such dramatic impacts (e.g. increased angiogenic potential; increased therapeutic efficacy in disease indications such as heart failure and GvHD) when present at pg/ml levels. Without wishing to be bound by any particular theory, the data provided by the present inventors, surprisingly suggest synergistic and/or more than additive effects of cytokines in the context of MLPSC culture expansion. For example, the present data indicates that provision of culture medium comprising TNF-alpha and IFN-gamma at concentrations <1 ng/ml can have profound impacts on MLPSCs culture expanded in the same and, that these impacts can be characterised based on levels of various angiogenic markers and/or clinical efficacy in patients.

Accordingly, the present inventors findings represent a significant advance in the art as they have shown how to prepare novel MLPSC populations that can direct improved therapeutic efficacy, in particular in the context of inflammation. These findings not only suggest that improved MLPSC populations can be provided through culture expansion in media supplemented with pro-inflammatory cytokines, they also indicate that relevant pro-inflammatory cytokines can be provided through culture expansion in medium supplemented with newborn serum. Accordingly, the present inventors findings underpin criteria for culture expansion of MLPSC in serum and serum free media.

### Example 8: MLPSC Isolation and Expansion

MLPSCs can be isolated using techniques such as STRO-3+ immunoselection of MPCs or density gradient separation of MSCs.

In general, relevant for bone marrow derived MLPCs, bone marrow (BM) is harvested from healthy normal adult volunteers (20-35 years old). Briefly, 40 ml of BM is aspirated from the posterior iliac crest into lithium-heparin anticoagulant-containing tubes.

BMMNC are prepared by density gradient separation using Lymphoprep (Nycomed Pharma, Oslo, Norway) as previously described (Zannettino et al. 1998). Following centrifugation at 400×g for 30 minutes at 4 C, the buffy layer is removed with a transfer pipette and washed three times in “HHF”, composed of Hank's balanced salt solution (HBSS; Life Technologies, Gaithersburg, MD), containing 5% fetal calf serum (FCS, CSL Limited, Victoria, Australia).

Relevant for immunoselection, STRO-3+ (or TNAP+) cells are subsequently isolated by magnetic activated cell sorting as previously described (Gronthos et al. 2003; Gronthos and Simmons 1995). Briefly, approximately 1-3×108 BMMNC are incubated in blocking buffer, consisting of 10% (v/v) normal rabbit serum in HHF for 20 minutes on ice. The cells are incubated with 200 ul of a 10 ug/ml solution of STRO-3 mAb in blocking buffer for 1 hour on ice. The cells are subsequently washed twice in HHF by centrifugation at 400×g. A 1/50 dilution of goat anti-mouse-biotin (Southern Biotechnology Associates, Birmingham, UK) in HHF buffer is added and the cells incubated for 1 hour on ice. Cells are washed twice in MACS buffer (Ca2+- and Mn2+-free PBS supplemented with 1% BSA, 5 mM EDTA and 0.01% sodium azide) as above and resuspended in a final volume of 0.9 ml MACS buffer.

One hundred ul streptavidin microbeads (Miltenyi Biotec; Bergisch Gladbach, Germany) are added to the cell suspension and incubated on ice for 15 minutes. The cell suspension is washed twice and resuspended in 0.5 ml of MACS buffer and subsequently loaded onto a mini MACS column (MS Columns, Miltenyi Biotec), and washed three times with 0.5 ml MACS buffer to retrieve the cells which did not bind the STRO-3 mAb (deposited on 19 Dec. 2005 with American Type Culture Collection (ATCC) under accession number PTA-7282—see International Publication No. WO 2006/108229). After addition of a further 1 ml MACS buffer, the column is removed from the magnet and the TNAP+ cells are isolated by positive pressure. An aliquot of cells from each fraction can be stained with streptavidin-FITC and the purity assessed by flow cytometry.

Alternatively, MSCs may be expanded from BMMNC using plastic adherence techniques. For example, bone marrow mononuclear cells can be isolated using ficoll-hypaque and placed into two T175 flasks with 50 ml per flask of culture expansion medium which includes alpha modified MEM (αMEM) containing gentamycin, glutamine (2 mM) and 10% (v/v) fetal bovine serum (FBS).

Cells are cultured for 2-3 days in 37° C., 5% CO2 at which time the non-adherent cells are removed; the remaining adherent cells are continually cultured until cell confluence reaches 70% or higher (7-10 days), and then the cells are trypsinized and replaced in six T175 flasks with expansion medium.

1. A composition comprising:
(i) a culture-expanded population of mesenchymal lineage precursor or stem cells (MLPSCs), wherein the MLPSCs have been culture expanded in media containing:
IFN-gamma and/or TNF-alpha; and/or,
one or more pro-inflammatory cytokines selected from the group consisting of IL-6; IL-8; IL-17A; MCP-1; MIP-1-alpha; MIP-1-beta; IP-10.

2. The composition of claim 1, wherein the media contains three or more pro-inflammatory cytokines.

3. The composition of claim 1 or claim 2, wherein the media contains two or more pro-inflammatory cytokines selected from the group consisting of IL-6; IL-8; IL-17A; MCP-1; MIP-1-alpha; MIP-1-beta; IP-10.

4. The composition according to any one of claims 1 to 3, wherein the media contains IL-6.

5. The composition according to any one of claims 1 to 4, wherein the media contains IL-8 and/or IL-17A.

6. The composition according to any one of claims 1 to 5, wherein the media contains IFN-gamma and TNF-alpha.

7. The composition according to any one of claims 1 to 6, wherein the level of IFN-gamma is <1 ng/ml, preferably <500 pg/ml, more preferably <100 pg/ml.

8. The composition according to any one of claims 1 to 7, wherein the level of TNF-alpha is <1 ng/ml, preferably <750 pg/ml, more preferably <400 pg/ml.

9. The composition according to any one of claims 1 to 8, wherein the media contains serum which comprises the pro-inflammatory cytokines.

10. The composition of claim 9, wherein the serum is newborn mammalian serum.

11. The composition of claim 9, wherein the serum is newborn calf serum.

12. The composition of claim 10 or claim 11, wherein the serum is obtained no more than 21 days after birth.

13. The composition according to any one of claims 1 to 12, wherein the media is characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

14. The composition according to any one of claims 1 to 12, wherein the media comprises at least 5% (v/v) newborn mammalian serum.

15. The composition according to any one of claims 1 to 6 or 9, wherein the media is serum free.

16. The composition according to any one of claims 1 to 15, wherein the MLPSCs express an increased level of angiogenin relative to a control population.

17. The composition according to any one of claims 1 to 16, wherein the MLPSCs induce increased levels of one or more of endothelial network formation, endothelial length, or endothelial branch length, relative to a control population.

18. The composition of claim 16 or claim 17, wherein the control population is a population of MLPSCs that have been culture expanded in a cell culture medium comprising 10% fetal serum.

19. The composition according to any one of claims 1 to 18, wherein the MLPSCs express a level of angiogenin greater than about 1200 pg/ml.

20. The composition according to any one of claims 1 to 19, wherein the MLPSCs express a level of SDF-1 greater than about 3000 pg/ml.

21. The composition according to any one of claims 1 to 20, wherein the MLPSCs express a level of VEGF greater than about 3200 pg/ml.

22. The composition according to any one of claims 1 to 21, wherein conditioned media from the MLPSCs induces endothelial network formation greater than about 0.12 mm²/mm².

23. The composition according to any one of claims 1 to 22, wherein conditioned media from the MLPSCs induces endothelial network length greater than about 5 mm²/mm².

24. The composition according to any one of claims 1 to 23, wherein conditioned media from the MLPSCs induces endothelial branch length greater than about 15 1/mm^{2.}

25. A cryopreserved composition comprising:
(i) a culture-expanded population of mesenchymal lineage precursor or stem cells (MLPSCs), wherein the MLPSCs have been culture expanded in media containing newborn mammalian serum obtained no more than 21 days after birth; and,
(iii) a cryopreservative.

26. The composition of claim 25, wherein the MLPSCs are cryopreserved at least twice.

27. An in vitro method for pre-licensing human mesenchymal lineage precursor or stem cells (MLPSCs), the method comprising culturing the MLPSCs in media containing:
i. IFN-gamma and/or TNF-alpha; and/or, one or more pro-inflammatory cytokines selected from the group consisting of IL-6; IL-8; IL-17A; MCP-1; MIP-1-alpha; MIP-1-beta; IP-10; and/or,
ii. newborn mammalian serum obtained no more than 21 days after birth.

28. The method of claim 27, wherein the media contains three or more pro-inflammatory cytokines.

29. The method of claim 27 or claim 28, wherein the media contains two or more pro-inflammatory cytokines selected from the group consisting of IL-6; IL-8; IL-17A; MCP-1; MIP-1-alpha; MIP-1-beta; IP-10.

30. The method according to any one of claims 27 to 29, wherein the media contains IL-6.

31. The method according to any one of claims 27 to 30, wherein the media contains IL-8 and/or IL-17A.

32. The method according to any one of claims 27 to 31, wherein the media contains IFN-gamma and TNF-alpha.

33. The method according to any one of claims 27 to 32, wherein the media contains serum which comprises the pro-inflammatory cytokines.

34. The method of claim 33, wherein the serum is newborn mammalian serum.

35. The method of claim 34, wherein the serum is newborn calf serum.

36. The method of claim 34 or claim 35, wherein the serum is obtained no more than 21 days after birth.

37. The method according to any one of claims 27 to 36, wherein the media is characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

38. The method according to any one of claims 27 to 37, wherein the media comprises at least 5% (v/v) newborn mammalian serum.

39. The method according to any one of claims 27 to 32 or 37, wherein the media is serum free.

40. A composition produced by the method according to any one of claims 27 to 39.

41. The composition or method according to any one of claims 7 to 40, wherein the newborn serum is postnatal day 1 to postnatal day 7 newborn serum.

42. The composition or method according to any one of claims 7 to 40, wherein the serum is at a concentration of about 2% (v/v) to about 12% (v/v).

43. The composition or method according to any one of claims 7 to 40, wherein the concentration of serum is about 5% (v/v).

44. The composition or method according to any one of claims 7 to 40, wherein the concentration of serum is about 10% (v/v).

45. The composition or method according to any one of claims 7 to 44, wherein the media comprises a fetal and a newborn serum from the same species and the ratio of the concentration of the fetal serum to the concentration of the newborn serum is 1:1 or lower.

46. The composition or method according to claim 45, wherein the concentration of the fetal serum and the concentration of newborn serum are each 5% (v/v).

47. The composition or method according to claim 45, wherein the concentration of the fetal serum is lower than that of the newborn serum.

48. The composition or method according to any one of claims 1 to 47, wherein the MLPSCs are maintained in an undifferentiated state.

49. The composition or method according to any one of claims 1 to 48, wherein the MLPSCs are human mesenchymal stem cells (hMSCs).

50. The composition or method according to any one of claims 1 to 49, wherein the MLPSCs are culture-expanded from a population of STRO-1⁺ multipotential cells.

51. A media for culturing MLPSCs, the media comprising:
IFN-gamma and/or TNF-alpha; and,
one or more pro-inflammatory cytokines, wherein the pro-inflammatory cytokines are selected from the group consisting of IL-6; IL-8; IL-17A; MCP-1; MIP-1-alpha; MIP-1-beta; IP-10.

52. The media of claim 51, wherein the media contains three or more pro-inflammatory cytokines.

53. The media of claim 51 or claim 52, wherein the media contains two or more pro-inflammatory cytokines selected from the group consisting of IL-6; IL-8; IL-17A; MCP-1; MIP-1-alpha; MIP-1-beta; IP-10.

54. The media according to any one of claims 51 to 53, wherein the media contains IL-6.

55. The media according to any one of claims 51 to 54, wherein the media contains IL-8 and/or IL-17A.

56. The media according to any one of claims 51 to 55, wherein the media contains IFN-gamma and TNF-alpha.

57. The media according to any one of claims 51 to 56, wherein the media contains serum which comprises the pro-inflammatory cytokines.

58. The media of claim 57, wherein the serum is newborn mammalian serum.

59. The media of claim 57, wherein the serum is newborn calf serum.

60. The media of claim 58 or claim 59, wherein the serum is obtained no more than 21 days after birth.

61. The media according to any one of claims 57 to 60, wherein the serum is characterised by one or more or all of the following:
i. a level of IFN-gamma greater than 10 pg/ml;
ii. a level of TNF-alpha greater than 20 pg/ml;
iii. a level of IL-6 greater than 30 pg/ml;
iv. a level of IL-8 greater than 5,000 pg/ml;
v. a level of IL-17A greater than 2 pg/ml;
vi. a level of MCP-1 greater than 30 pg/ml;
vii. a level of MIP-1-alpha greater than 5 pg/ml;
viii. a level of MIP-1-beta greater than 30 pg/ml;
ix. a level of IP-10 greater than 5,000 pg/ml.

62. The media according to any one of claims 57 to 61, wherein the media comprises at least 5% (v/v) newborn mammalian serum.

63. The media according to any one of claims 57 to 61, wherein the media comprises 5% (v/v) newborn mammalian serum.

64. The media according to any one of claims 57 to 56, wherein the media is serum free.
